(19) United States
(12) Patent Application Publication
(10) Pub. No.: US 2025/0302867 A1
(43) Pub. Date: Oct. 2, 2025

(54) METHOD FOR TREATING STRESS URINARY INCONTINENCE

(71) Applicant: ATHENA UROLOGIC, LLC, White Plains, NY (US)

(72) Inventor: Nicole FLEISCHMANN, White Plains, NY (US)

(21) Appl. No.: 18/864,227

(22) PCT Filed: Apr. 28, 2023
(86) PCT No.: PCT/US2023/066365

§ 371 Date: Nov. 8, 2024

Related U.S. Application Data
(60) Provisional application No. 63/339,913, filed on May 9, 2022.
(60) Provisional application No. 63/386,047, filed on Dec. 5, 2022.

(51) Int. Cl. A61K 31/785; A61K 9/00

(52) U.S. Cl. CPC A61K 31/785; A61K 9/0019; A61K 9/0034

(57) ABSTRACT

The present disclosure relates to method of treating stress urinary incontinence in a female subject, with endoscopic reconstruction of the external urinary sphincter using injection of a bulking agent. The reconstruction of the external urethral sphincter may be performed by injecting a bulking agent into the external urethral sphincter of the subject. The bulking agent may be administered into to a circumference of the external urethral sphincter. The method may also include inserting a portion of an endoscopic video camera into a urethra and visually locating a urethral crest. A bulking agent may be injected through a needle into an external urethral sphincter of the subject at a location to create a visual seal between the urethral crest and a compressor urethrae of the subject, and to create a visual seal between the urethral crest and a urethrovaginal sphincter of the subject.

### CROSS-REFERENCE TO RELATED APPLICATIONS

The present International Patent application claims priority to U.S. Provisional Patent Application No. 63/339,913 filed on May 9, 2022, and U.S. Provisional Patent Application No. 63/386,047 filed on Dec. 5, 2022, the entirety of the disclosures of which are incorporated herein by reference.

### FIELD OF THE INVENTION

The present invention generally relates to the noninvasive treatment of stress urinary incontinence, and more particularly, to a method for injecting a biocompatible bulking agent into tissue of a subject for treating stress urinary incontinence.

### BACKGROUND

Urinary incontinence is the unintentional loss of urine. Stress urinary incontinence (SUI) is the unintentional loss of urine when physical movement or activity (such as coughing, laughing, sneezing, exercise or heavy lifting) puts pressure (stress) on the bladder, causing urine to leak. It is a common condition that reportedly affects over 28 million female adults in the United States. This condition can have a significant impact on daily life, affecting activities, relationships and emotional well-being. It can occur at any stage of life, and pelvic disorders from childbirth, pelvic surgery and aging increase the risk of incontinence.

After attempts at conservative treatments which may include pelvic floor exercises, biofeedback, behavioral and dietary management, continence rings and/or plugs and pads, the current most common and preferred method of treating stress incontinence by physicians is midurethral sling surgery (MUS). The procedure has many iterations but usually involves the implantation of a strip of polypropylene mesh under the urethral wall. Although 85-90% effective in curing the condition, these surgeries involve a relatively lengthy recovery time and like any surgery, come with risks. Albeit not common, potential complications of such surgery include temporary difficulty urinating and incomplete bladder emptying, development of overactive bladder, which could include urge incontinence, bleeding and clot formation, nerve damage, erosion of the mesh implant through the vaginal wall or urinary tract, pain, urinary tract infection and difficult or painful sexual intercourse. Many women are reticent to undergo these procedures. Indeed, even in experienced users' hands, serious complications are believed to be as high as 13.8% and less serious complications as high as 36.9%. Moreover, with the exception of mesh complications, the complication rates of the MUS are actually lower than that of traditional procedures such as autologous fascial sling and retropubic urethropexy.

Stress urinary incontinence remains grossly undertreated compared to other non-life-threatening, quality of life disease conditions, such as cataracts. Certain population-based studies have shown that a staggering number of roughly 25 million people in the United States suffer from SUI. However, only 150 to 170 thousand procedures for SUI, the majority of which are MUS, are performed annually in the United States. In contrast, every year, over 3 million Medicare beneficiaries receive surgery for their cataracts, a condition which has a similar prevalence. The most likely explanation for this discrepancy is the superior risk/benefit profile of cataract surgery compared to the MUS. No conventional surgical procedure for SUI exists with such optimal statistics. Accordingly, medical providers often counsel women that intervention should only be performed if conservative therapy such as Kegel exercises have been tried, and if there is significant discomfort and interference with quality of life. Furthermore, many women believe that incontinence is a normal part of life, which compounds their reluctance to seek help. Consequently, urine leakage remains a significant health problem that has considerable deleterious effects, including those both physically and mentally, such as embarrassment, social isolation, and depression.

A minimally invasive alternative to the surgical procedure is the injection of a bulking agent into the proximal urethra, in the region of the internal urinary sphincter (IUS). Although such injections are less invasive and involve less risk of serious complications than surgery, they are less effective than MUS and often do not fully alleviate the urinary incontinence. Traditional bulking agents are in the class of particulate compounds, solid microparticles in an absorbable liquid or gel carrier which can expand the submucosal tissues to narrow the opening of the bladder neck causing a reduction in urine leakage. Over time, the bulking effect is lost due to absorption of the carrier gel leaving only the inflammatory microparticles remaining. In these cases, incontinence returns and the injection needs to be repeated. Furthermore, the remaining particles in all of these formulations are immunogenic and can lead to rare complications such as abscess formation and urethral erosion requiring implant removal. Due to these risks, and the limited success and longevity of such treatments, the use of bulking agents to treat stress incontinence in the US has been sporadic and reserved for only specific cases. More recently, use of polyacrylamide hydrogel (PAHG) (Bulkamid® (Axonics, Contura)) a nonparticulate bulking agent has gained popularity for use in this approach. Because there is no carrier gel to dissolve and no inflammatory particles, PAHG does not lose volume over time and causes no inflammation in the urethral tissues.

Unfortunately, cure rates with current methods of Bulkamid® injection in the proximal urethra are not much better than traditional particulate bulking agents. Even still, many women prefer injectable therapies for their leakage because of an acceptable safety profile and negligible downtime, even though they realize the outcome is likely inferior. Yet, because urethral bulking agents have failed to be effective in curing the condition, clinicians infrequently offer them as a treatment option. The other obstacle is the poor reimbursement for such procedures in an office-setting, which gives little incentive for providers to improve their technique and increase their utilization. The use of a non-particulate, non-inflammatory and durable material, such as PAHG, with a standardized delivery system 2 for proximal urethral bulking, is illustrated in FIG. 4. Unfortunately, the data on PAHG is not much improved over other known urethral bulking agents. For instance, in a randomized controlled study, at one year, PAHG had a 67% objective cure rate compared to 95% rate for tension-free vaginal tape surgery (TVT). At seven years, only 66% of patients felt either improvement or were cured with Bulkamid®.

Therefore, there remains a need for developing novel strategies for treating stress urinary incontinence that are effective and women find to have an acceptable risk profile and recovery period. Moreover, there is a clear and substantial need for an improved method involving the placement of a bulking agent at the correct anatomical location of the female sphincter mechanism in the treatment of stress urinary incontinence.

### SUMMARY OF THE INVENTION

The present disclosure provides a minimally invasive method that is superior to the current minimally invasive methods of treating stress urinary incontinence and is at least as successful as the surgical methods (e.g., MUS). The method described herein results in 80-90% cure rates that represent a vast improvement over the standard technique cure rates of less than 50%.

In certain aspects, the present disclosure provides methods of treating stress urinary incontinence in a female subject, comprising administering an agent into the external urethral sphincter of the subject. In some embodiments, the agent is injected into the external urethral sphincter of the subject. In some embodiments, the agent is administered into the external urethral sphincter of the subject at a position that is about 2-3 cm from the bladder neck. In some embodiments, the agent is administered into to the circumference of the external urethral sphincter. In some embodiments, the agent is administered as a single injection. In some embodiments, the agent is administered as multiple injections. In some embodiments, the agent is a bulking agent. In some embodiments, the bulking agent is a hydrogel. In some embodiments, the hydrogel is polyacrylamide hydrogel (PAHG). In some embodiments, the polyacrylamide is cross-linked polyacrylamide.

In some embodiments, the PAHG comprises about 1% to about 10% (w/w) polyacrylamide. In some embodiments, the PAHG comprises about 2% (w/w) polyacrylamide. In some embodiments, the PAHG comprises 2% (w/w) polyacrylamide. In some embodiments, the PAHG comprises about 2.5% (w/w) polyacrylamide. In some embodiments, the d PAHG comprises 2.5% (w/w) polyacrylamide. In some embodiments, the PAHG comprises about 3% (w/w) polyacrylamide. In some embodiments, the PAHG comprises 3% (w/w) polyacrylamide. In some embodiments, the PAHG comprises about 5% (w/w) polyacrylamide. In some embodiments, the PAHG comprises 5% (w/w) polyacrylamide.

In some embodiments, about 1 ml to about 10 ml of PAHG is administered into the external urethral sphincter of the subject. In some embodiments, about 2 ml to about 4 ml of PAHG is administered into the external urethral sphincter of the subject. In some embodiments, an amount of PAHG sufficient to reconstruct the external urethral sphincter is administered into the external urethral sphincter of the subject.

In certain aspects, the present disclosure provides methods of treating stress urinary incontinence in a female subject, comprising reconstructing the external urethral sphincter by injecting an agent into the external urethral sphincter of the subject. In some embodiments, the agent is injected into the external urethral sphincter of the subject. In some embodiments, the agent is administered into the external urethral sphincter of the subject at a position that is about 2-3 cm from the bladder neck. In some embodiments, the agent is administered into to the circumference of the external urethral sphincter.

In some embodiments, the agent is administered as a single injection. In some embodiments, the agent is administered as multiple injections.

In some embodiments, the agent is a bulking agent. In some embodiments, the bulking agent is a hydrogel. In some embodiments, the hydrogel is polyacrylamide hydrogel (PAHG). In some embodiments, the polyacrylamide is cross-linked polyacrylamide. In some embodiments, said PAHG comprises about 1% to about 10% (w/w) polyacrylamide. In some embodiments, said PAHG comprises about 2% (w/w) polyacrylamide. In some embodiments, said PAHG comprises 2% (w/w) polyacrylamide. In some embodiments, said PAHG comprises about 2.5% (w/w) polyacrylamide. In some embodiments, said PAHG comprises 2.5% (w/w) polyacrylamide. In some embodiments, said PAHG comprises about 3% (w/w) polyacrylamide. In some embodiments, said PAHG comprises 3% (w/w) polyacrylamide. In some embodiments, said PAHG comprises about 5% (w/w) polyacrylamide. In some embodiments, said PAHG comprises 5% (w/w) polyacrylamide.

In some embodiments, about 1 to about 10 ml of PAHG in administered into the external urethral sphincter of the subject. In some embodiments, about 2 to about 4 ml of PAHG in administered into the external urethral sphincter of the subject. In some embodiments, an amount of PAHG sufficient to reconstruct the external urethral sphincter is administered into the external urethral sphincter of the subject.

In certain aspects, the present invention provides methods of treating stress urinary incontinence in a female subject, comprising performing an endoscopic reconstruction of the external urethral sphincter by injecting an agent into the external urethral sphincter of the subject. In some embodiments, the agent is injected into the external urethral sphincter of the subject. In some embodiments, the agent is administered into the external urethral sphincter of the subject at a position that is about 2-3 cm from the bladder neck. In some embodiments, the agent is administered into to the circumference of the external urethral sphincter. In some embodiments, the agent is administered as a single injection. In some embodiments, the agent is administered as multiple injections.

In some embodiments, the agent is a bulking agent. In some embodiments, the bulking agent is a hydrogel. In some embodiments, the hydrogel is polyacrylamide hydrogel (PAHG). In some embodiments, the polyacrylamide is cross-linked polyacrylamide. In some embodiments, said PAHG comprises about 1% to about 10% (w/w) polyacrylamide. In some embodiments, said PAHG comprises about 2% (w/w) polyacrylamide. In some embodiments, said PAHG comprises 2% (w/w) polyacrylamide. In some embodiments, said PAHG comprises about 2.5% (w/w) polyacrylamide. In some embodiments, said PAHG comprises 2.5% (w/w) polyacrylamide. In some embodiments, said PAHG comprises about 3% (w/w) polyacrylamide. In some embodiments, said PAHG comprises 3% (w/w) polyacrylamide. In some embodiments, said PAHG comprises about 5% (w/w) polyacrylamide. In some embodiments, said PAHG comprises 5% (w/w) polyacrylamide.

In some embodiments, about 1 to about 10 ml of PAHG in administered into the external urethral sphincter of the subject. In some embodiments, about 2 to about 4 ml of PAHG in administered into the external urethral sphincter of the subject. In some embodiments, an amount of PAHG sufficient to reconstruct the external urethral sphincter is administered into the external urethral sphincter of the subject.

In certain aspects, the present disclosure provides methods of improving longevity of a treatment of treating stress urinary incontinence of a subject, comprising: performing any of the methods as disclosed above; and providing instructions to the subject for abstaining from pelvic muscle clenching and abdominal straining while voiding and/or defecating after the agent has been administered, thereby preventing dislodging of the administered agent. In some embodiments, the subject is a human.

In certain aspects, the present disclosure provides methods for treating stress urinary incontinence, the method comprising: inserting a portion of an endoscopic video camera into a urethra of a subject and visually locating a urethral crest of the subject; retracting the endoscopic video camera to an external meatus of the subject; injecting an agent through a needle into an external urethral sphincter of the subject at a location to create a visual seal between the urethral crest and a compressor urethrae of the subject, and to create a visual seal between the urethral crest and a urethrovaginal sphincter of the subject. In some embodiments, the method further comprises milking a submucosal tissue of the subject by placing a downward force on the urethra to reveal the urethral crest. In some embodiments, the endoscopic video camera comprises a cystoscope including a display screen. In some embodiments, the cystoscope includes a zero-degree lens. In some embodiments, a location of the urethral crest is displayed on an inferior portion the display screen. In some embodiments, the method further comprises irrigating and draining a bladder of the subject. In some embodiments, the agent is administered as a single injection. In some embodiments, the agent is administered as multiple injections. In some embodiments, the agent is a bulking agent. In some embodiments, the bulking agent is a hydrogel. In some embodiments, the hydrogel is polyacrylamide hydrogel (PAHG).

In certain aspects the present invention provides kits for use in treating stress urinary incontinence in a female subject, the kit comprising a dispensing device and an agent for administering into the external urethral sphincter of the subject via the dispensing device. In some embodiments, the agent comprises a bulking agent. In some embodiments, the bulking agent comprises a hydrogel. In some embodiments, the bulking agent comprises a polyacrylamide hydrogel (PAHG). In some embodiments, the agent is preloaded into the dispensing device. In some embodiments, the dispensing device comprises a syringe. In some embodiments, the agent is sterile packaged in a container or vial. In some embodiments, the kit further comprises one or more sets of instructions for use.

There has thus been outlined certain embodiments of the present invention in order that the detailed description thereof herein may be better understood, and in order that the present contribution to the art may be better appreciated. There are additional embodiments of the present invention that will be described below, and which form the subject matter of the claims appended hereto.

In this respect, before explaining at least one aspect of the method for treating stress urinary incontinence in detail, it is to be understood that the apparatus and method are not limited in their application to the details of construction and to the arrangements of the components set forth in the following description or illustrated in the drawings. The apparatus and method are capable of aspects in addition to those described, and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the present invention. It is important, therefore, that the claims be regarded as including such equivalent constructions insofar as they do not depart from the spirit and scope of the invention.

### BRIEF DESCRIPTION OF THE DRAWINGS

In order that the present invention may be readily understood, aspects of the invention are illustrated by way of examples in the accompanying drawings, in which like parts are referred to with like reference numerals throughout. The file of this patent or application contains at least one drawing/photograph executed in color. Copies of this patent or patent application publication with color drawing(s)/photograph(s) will be provided by the Office upon request and payment of the necessary fee.

FIG. 1 is a diagram depicting the female pelvic anatomy and urethral sphincter complex.

FIG. 2 is a diagram depicting the continence mechanism at midurethra including EUS.

FIG. 3 is a diagram depicting a view of the EUS/IUS through the cystoscope and a comparison of the EUS technique with the standard technique (prior minimally invasive technique).

FIG. 4 depicts the administration of a non-particulate, non-inflammatory and durable material, such as PAHG, with a standardized delivery system for proximal urethral bulking.

FIG. 5 illustrates compressive forces of a first closure member in a urethra.

FIG. 6A illustrates a complete alignment of the distal EUS.

FIG. 6B illustrates a displacement of the EUS muscles.

FIG. 7 illustrates an intrinsic sphincter deficiency causing urine leakage from a misaligned sphincter muscle.

FIG. 8 illustrates muscle realignment with a bulking agent injection in accordance with the present invention.

FIG. 9 illustrates a sample view of a urethra through a urethroscope.

FIG. 10 illustrates cystoscopic reconstruction of the external sphincter in accordance with the present invention.

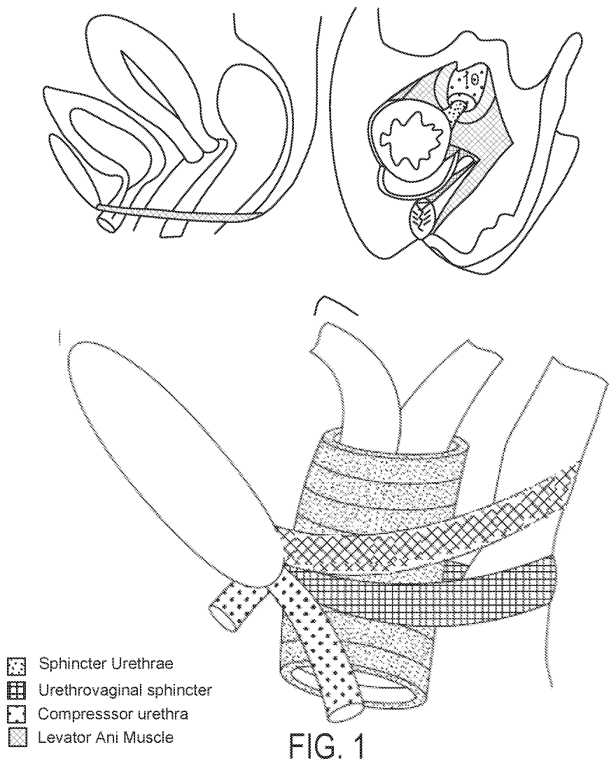

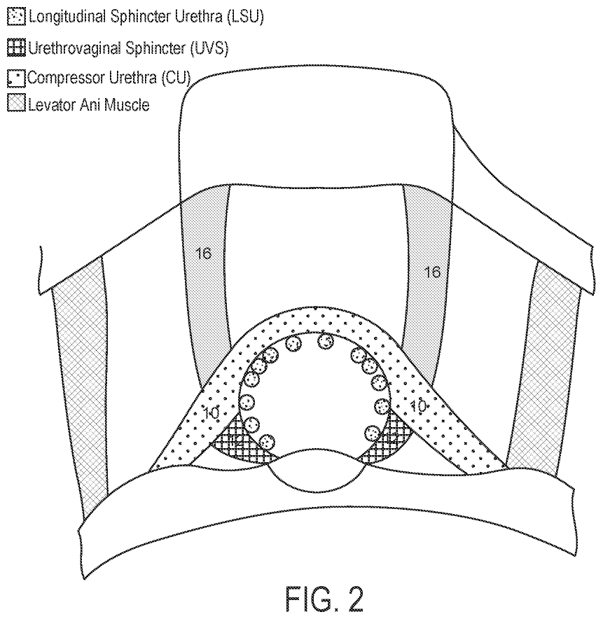

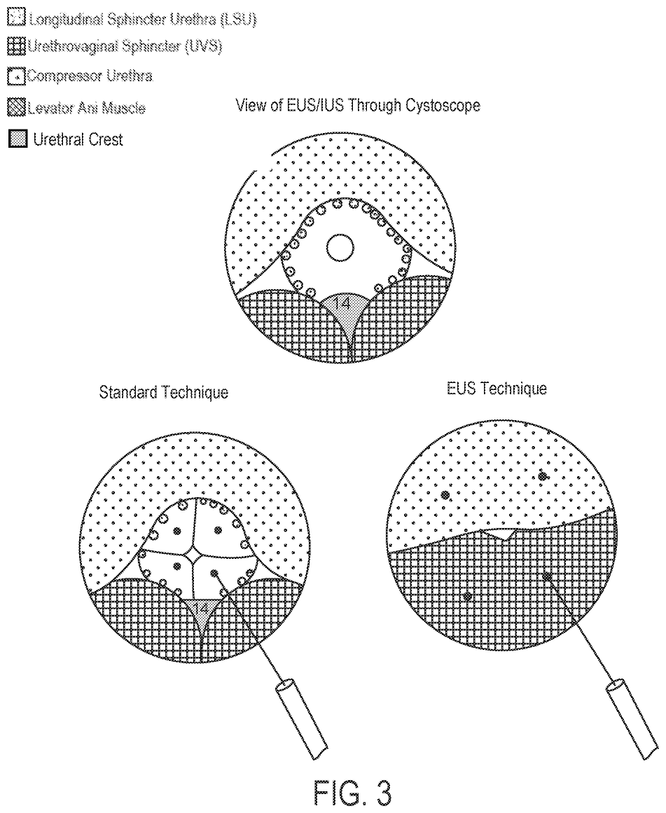

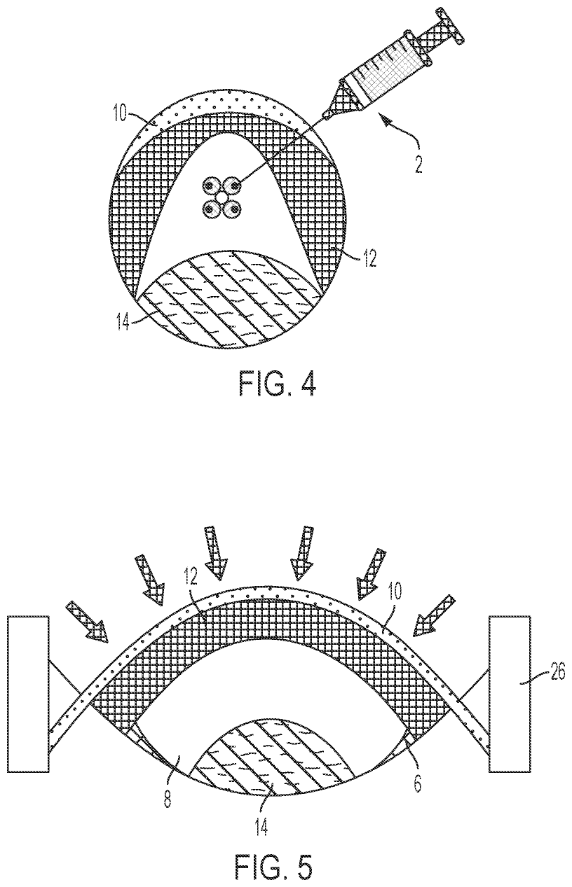

### DETAILED DESCRIPTION

### General

Practice of the methods, as well as preparation and use of the compositions disclosed herein employ, unless otherwise indicated, conventional techniques in chemistry, molecular biology, biochemistry, medicine and related fields as are within the skill of the art.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “subject” and “patient” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject.

The terms “treating” and “treatment” or variations thereof, as used herein, refer to reduction in severity and/or frequency of symptoms, elimination of symptoms and/or underlying cause, prevention of the occurrence of symptoms and/or their underlying cause, delaying the occurrence of symptoms and/or their underlying cause, and improvement or remediation of damage. The treatment may improve the quality of life.

An “effective dose” or “effective amount,” as used herein, refers to a dose and/or amount of the composition given to a subject as disclosed herein, that can help treat or prevent the occurrence of symptoms.

### Description

For decades, investigators have strived to determine which anatomic structure, urethral closure mechanism or pelvic floor muscle support, is the most responsible for maintaining continence in women with SUI. Compressive forces of the first closure mechanism are shown in FIG. 5. In particular, the vaginal hammock, a condensation of fascia between the bladder neck and distal urethra, creates a backboard for compression of the proximal urethra 7 during increased intra-abdominal pressure. Further, mid-urethral support is important and forms the rationale for TVT, since the active urethral closure mechanism is made possible by several anatomical support structures, the most important of which are the pubourethral ligaments (PUL) 16 that attach the pubic bone 24 to the midurethra. The integrity of the PUL 16 allows the distal muscles of the EUS—the compressor urethrae (CU) 10, and the urethrovaginal sphincter (UVS) 12, to directly approximate with the urethral crest (UC) 14, sealing off the urethral opening. The UC 14, also known by its Latin name, crista urethralis, is embryologically analogous to the veromontanum in males and has properties similar to cavernosal tissue. The CU 10 and UVS 12 are fast-twitch fibers that are continuous with the distal border of the slow twitch fibers of the striated sphincter urethrae (SSU) 18. The CU 10 passes over the urethra 8 to insert into the urogenital diaphragm at the pubic ramus. The UVS 12 surrounds both the distal urethra and the vagina 6. It has no bony attachments but is convergent at the ventral side with the CU 10, forming one complex which extends laterally down the sides of the urethra to the perineal body.

When the CU 10 and UVS 12 contract, they exert a downward force on the lumen of the urethra, compressing the tissues of the urethral crest and creating a strong seal for continence, as depicted in FIG. 5. The configuration is important in that it implies that the closure of the urethra by the EUS is done slit-wise by compressing the ventral part against the dorsal part instead of closing in a purely circular way. This mechanism is referred to as a first closure mechanism, and its impairment is responsible for 50% of SUI. In addition, a second closure mechanism relies on both functioning PULs as well as apical support of the vaginal wall. Poor proximal urethra support, as exists when there is significant apical prolapse, prevents the necessary backward motion of the levator plate which then fails to stretch the proximal urethra 7 at the zone of critical elasticity (ZCE), thus impairing bladder neck closure. It has been postulated that an intact first closure mechanism is an important prerequisite for the second closure mechanism to properly function.

The term intrinsic sphincter deficiency (ISD) was originally coined in urology literature in the 1970s and was used to describe a severe form of stress urinary incontinence characterized by a fixed open vesical neck without descent of the bladder base. The main continence mechanism was believed to be at the bladder neck or internal urinary sphincter (IUS) and that patients who failed retropubic urethropexy required a pubovaginal sling (PVS), which worked by compressing the urethra and maintaining continence even in patients with poor functioning outlets. From this reasoning, UBAs, such as bovine collagen (Contigen®), were injected in the IUS with the idea of improving urethral resistance during periods of increased abdominal pressure through endoscopic submucosal tissue bulking. There is still a belief that UBAs are only appropriate in patients with classic signs of ISD (leakage in cases of a well-supported bladder neck, leak point pressure below 60 cm H₂O, MUCP below 20 cm H₂O, open bladder neck on VCUG) as opposed to patients with urethral hypermobility (UH). Over the years, numerous UBA products have been developed. For instance, four injectable agents approved for this procedure include Macroplastique®, Coaptite®, Durasphere® and Bulkamid®. As previously described above, Bulkamid® is PAHG that forms its own class of nonparticulates which incorporate and do not biodegrade.

The anatomy of the female urethra is complex, with multiple areas that could contribute to urethral seal, including the external urinary sphincter. Given that the outcomes of UBAs using a proximal submucosal injection technique are inferior to surgery, the present method provides an innovative injection technique for the injection material in order to improve success rates and meet patients' demand for a noninvasive solution. The procedure of the present invention improves the seal of the striated muscle of the external urinary sphincter, under direct vision, with an injectable agent that is biologically compatible. One of the reasons the technique of the present invention has not previously been performed before is at least partly because the EUS is not an obvious structure seen on female cystourethroscopy and is therebefore not routinely identified. However, its importance in creating the seal that prevents SUI may be critical, and thus may explain why support of this structure with a sling procedure increases its function in patients with urethral hypermobility.

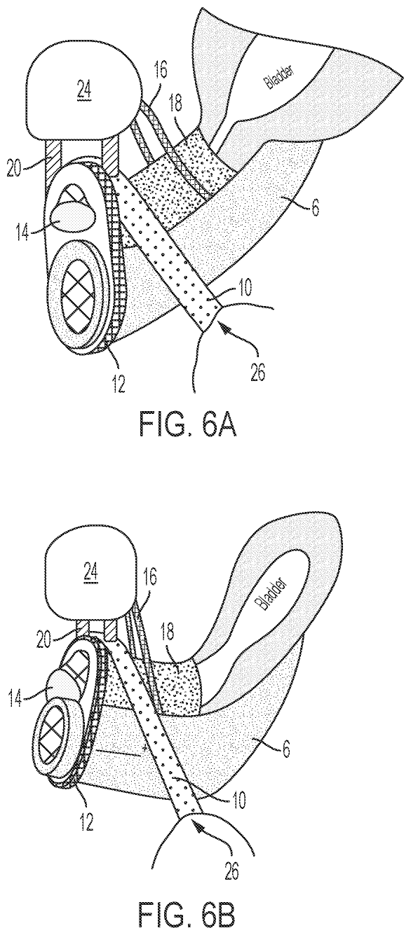

The postulate that EUS misalignment is a cause for SUI in women with UH is a novel concept that overlaps well with the integral theory. In actuality, it is a further explanation of the first closure mechanism. Under normal circumstances, there is complete alignment (zero-degree displacement) of the components of the distal EUS so that the downward contraction of the EUS muscles onto the UC causes a firm seal, as illustrated in FIG. 6A. Unlike the PUL 16, the EUL 20 remains relatively unharmed by childbirth injury and continues to affix the distal EUS muscles to the pubic bone 24, even in cases of severe vaginal laxity. This contributes to the rotational movement of the external meatus around the EUL 20 as abdominal pressure increases. Purportedly, the MUS procedure works to recreate the PUL 16 and prevent urethral hypermobility, yet its role in EUS function has not been well delineated. It is believed the MUS, in effect, stabilizes the EUS by preventing a distal sphincter misalignment and incomplete seal of the EUS mechanism.

The mechanics of sphincter misalignment are depicted in FIG. 6B, in which the CU 10 and UVS 12 have separate inferior attachments. The CU 10 is attached to the bone of the ischial ramus 26 whereas the UVS 12 only relates to the mobile vaginal wall. During urethral decent, there is a separation of the points of attachment of these two structures, such that the CU 10 stays fixed to the ramus 26 but the UVS 12 moves downward with the vaginal wall. Due to the location of the UC 14 at the distal anterior vaginal wall, and because the direction of the compressive force from the EUS muscles stays fixed in line with the EUL 20, the displacement causes the EUS muscles to compress a more proximal and less dense portion of the posterior urethra than the cavernosal-like structure of the UC 14. This displacement impairs the ability of the EUS to create a proper seal, thus causing SUI.

In the current model, poor apical support would also compromise EUS alignment, and therefore, compromise the function of the first closure mechanism. By elevating the apical anterior vaginal wall in patients who have significant bladder neck hypermobility, an abdominal sacral colpopexy or uterosacral ligament suspension would reduce the separation of the UVS 12 and CU 10 and pull the urethral crest back into approximation with the EUS muscles. It has been observed that in patients with prolapse and SUI undergoing MUS, patients with tighter USL repairs had better continence outcomes than those with loose USL repairs. Apical support facilitating EUS closure seems counterintuitive when considering the similarly appearing but entirely distinct mechanism of unmasking occult stress incontinence in patients who have apical prolapse repairs. In the latter scenario, prolapse correction unobstructs the bladder neck, increasing the flow to, and unveiling a weakly functioning, EUS. Therefore, EUS closure is also dependent on substantial tissues for creating a seal. Realignment of the EUS through vaginal suspension alone will not work to cure incontinence in the setting of striated muscle atrophy as seen with aging, estrogen deficiency, urethral scarring, fibrosis or denervation of the EUS muscles. In this sense, an injection may work in all patients with SUI, either from EUS misalignment, EUS muscle atrophy or a combination of both.

The present disclosure provides a minimally invasive method that is superior to the current minimally invasive methods of treating stress urinary incontinence and is at least as effective at alleviating the incontinence as the surgical methods. The method of the present disclosure includes Cystoscopic Reconstruction/Realignment of External Sphincter Technique (CREST Procedure). The method disclosed herein results in unexpectedly high rates of successfully treating stress urinary incontinence with no or minimal adverse events. There is less urinary retention and urinary tract infection due to the lack of obstructive process and the dynamic nature of the external urethral sphincter complex, which is unexpected because of previous concerns that particulate agents as opposed to a hydrogel injected into the external urethral sphincter could damage the sphincter and/or worsen sphincter function because injecting an inflammatory particulate substance into the sphincter would elicit a fibrotic process.

In one aspect, the disclosure provides a method of treating stress urinary incontinence in a female subject, comprising administering an agent into the EUS of the female subject. In some embodiments, the disclosure provides a method of treating stress urinary incontinence in a female subject, comprising a reconstruction of a deficient external urethral sphincter (EUS) by administering an agent into the EUS of the subject. In some embodiments, the disclosure provides a method of treating stress urinary incontinence in a female subject, comprising an endoscopic reconstruction of a deficient external urethral sphincter (EUS) by administering an agent into the EUS of the subject. In some embodiments, the disclosure provides a method of treating stress urinary incontinence in a female subject, comprising an endoscopic reconstruction of a deficient external urethral sphincter (EUS) by administering an agent into the EUS of the subject. In some embodiments, the method comprises injecting the agent into the external urethral sphincter of the subject. In some embodiments, the agent is injected into the external urethral sphincter of the subject at a position that is about 2-3 cm from the bladder neck. In some embodiments, the agent is injected into to the circumference of the external urethral sphincter (EUS). In some embodiments, the method comprises multiple injections.

In some embodiments, the disclosure provides a method of treating stress urinary incontinence in a female subject, comprising administering a bulking agent into the EUS of the female subject. In some embodiments, the disclosure provides a method of treating stress urinary incontinence in a female subject, comprising a reconstruction of a deficient external urethral sphincter (EUS) by administering a bulking agent into the EUS of the subject. In some embodiments, the disclosure provides a method of treating stress urinary incontinence in a female subject, comprising an endoscopic reconstruction of a deficient external urethral sphincter (EUS) by administering a bulking agent into the EUS of the subject. In some embodiments, the disclosure provides a method of treating stress urinary incontinence in a female subject, comprising an endoscopic reconstruction of a deficient external urethral sphincter (EUS) by administering a bulking agent into the EUS of the subject. In some embodiments, the method comprises injecting the bulking agent into the external urethral sphincter of the subject. In some embodiments, the bulking agent is injected into the external urethral sphincter of the subject at a position that is about 2-3 cm from the bladder neck. In some embodiments, the bulking agent is injected into to the circumference of the external urethral sphincter (EUS). In some embodiments, the method comprises multiple injections.

In some embodiments, the bulking agent is a hydrogel. In some embodiments, the hydrogel is a polyacrylamide hydrogel (PAHG). In some embodiments, the hydrogel is a hydrogel formed of one or more other biocompatible polymers such as collagen, gelatin, hyaluronic acid, polyethylene glycol, and the like. For example, in some embodiment, the hydrogel is one or more of a collagen hydrogel, a gelatin hydrogel, a hyaluronic acid hydrogel, a polyethylene glycol hydrogel (PEG), or one or more combinations thereof.

In some embodiments, the disclosure provides a method of treating stress urinary incontinence in a female subject, comprising administering a bulking agent into the external urethral sphincter of the subject. In some embodiments, the disclosure provides a method of treating stress urinary incontinence in a female subject, comprising reconstructing the external urethral sphincter by injecting bulking agent into the external urethral sphincter of the subject. In some embodiments, the disclosure provides a method of treating stress urinary incontinence in a female subject, comprising an endoscopic reconstruction of the external urethral sphincter by injecting bulking agent into the external urethral sphincter of the subject. In some embodiments, the method comprises injecting the bulking agent into the external urethral sphincter of the subject. In some embodiments, the bulking agent is injected into the external urethral sphincter of the subject at a position that is about 2-3 cm from the bladder neck. In some embodiments, the bulking is injected into to the circumference of the external urethral sphincter.

In some embodiments, the method comprises using multiple 1 ml pre-filled syringes of bulking agent, for example a hydrogel. In some embodiments, the method comprises using one pre-filled syringe of bulking agent. In some embodiments, the method comprises two pre-filled syringes of bulking agent. In some embodiments, the method comprises three pre-filled syringes of bulking agent. In some embodiments, the method comprises four pre-filled syringes of bulking agent. In some embodiments, the method comprises five pre-filled syringes of bulking agent. In some embodiments, the method comprises six pre-filled syringes of bulking agent. In some embodiments, the method comprises seven pre-filled syringes of bulking agent. In some embodiments, the method comprises eight pre-filled syringes of bulking agent. In some embodiments, the method comprises nine pre-filled syringes of bulking agent. In some embodiments, the method comprises ten pre-filled syringes of bulking agent.

In some embodiments, the agent is a bulking agent. In some embodiments, the bulking agent is a hydrogel. In some embodiments, the hydrogel is a biocompatible polymer. For example, in some embodiments the hydrogel is polyacrylamide, collagen, gelatin, polyethylene glycol (PEG), or one or more combinations thereof. In some embodiments, the hydrogel is a polyacrylamide hydrogel. In some embodiments, the polyacrylamide is cross-linked polyacrylamide. In some embodiments, the hydrogel is a collagen hydrogel. In some embodiments, the collagen is crosslinked collagen. In some embodiments, the hydrogel is a gelatin hydrogel. In some embodiments, the gelatin is crosslinked gelatin. In some embodiments, the hydrogel is a hyaluronic acid hydrogel. In some embodiments, the hyaluronic acid is crosslinked hyaluronic acid. In some embodiments, the hydrogel is a polyethylene glycol (PEG) hydrogel. In some embodiments, the PEG is crosslinked PEG. In some embodiments, the hydrogel is a hydrogel containing one or more biocompatible polymers including one or more combinations of one or more of polyacrylamide, collagen, gelatin, hyaluronic acid, and/or polyethylene glycol.

In some embodiments, the hydrogel comprises about 1% to about 10% (w/w) biocompatible polymer. In some embodiments, the hydrogel comprises about 1% to about 9% (w/w) biocompatible polymer. In some embodiments, the hydrogel comprises about 1% to about 8% (w/w) biocompatible polymer. In some embodiments, the hydrogel comprises about 1% to about 7.5% (w/w) biocompatible polymer. In some embodiments, the hydrogel comprises about 1% to about 7% (w/w) biocompatible polymer. In some embodiments, the hydrogel comprises about 1% to about 6% (w/w) biocompatible polymer. In some embodiments, the hydrogel comprises about 1% to about 5% (w/w) biocompatible polymer. In some embodiments, the hydrogel comprises about 1% to about 4% (w/w) biocompatible polymer. In some embodiments, the hydrogel comprises about 1% to about 3% (w/w) biocompatible polymer. In some embodiments, the hydrogel comprises about 1% to about 2.5% (w/w) biocompatible polymer. In some embodiments, the hydrogel comprises about 1% to about 2% (w/w) biocompatible polymer. In some embodiments, the hydrogel comprises about 2% to about 5% (w/w) biocompatible polymer. In some embodiments, the hydrogel comprises about 2.5% to about 5% (w/w) biocompatible polymer. In some embodiments, the hydrogel comprises about 2.5% to about 4% (w/w) biocompatible polymer. In some embodiments, the hydrogel comprises about 2.5% to about 3% (w/w) biocompatible polymer.

In some embodiments, the hydrogel comprises about 1% to about 10% (w/w) cross-linked biocompatible polymer. In some embodiments, the hydrogel comprises about 1% to about 9% (w/w) cross-linked biocompatible polymer. In some embodiments, the hydrogel comprises about 1% to about 8% (w/w) cross-linked biocompatible polymer. In some embodiments, the hydrogel comprises about 1% to about 7.5% (w/w) cross-linked biocompatible polymer. In some embodiments, the hydrogel comprises about 1% to about 7% (w/w) cross-linked biocompatible polymer. In some embodiments, the hydrogel comprises about 1% to about 6% (w/w) cross-linked biocompatible polymer. In some embodiments, the hydrogel comprises about 1% to about 5% (w/w) cross-linked biocompatible polymer. In some embodiments, the hydrogel comprises about 1% to about 4% (w/w) cross-linked biocompatible polymer. In some embodiments, the hydrogel comprises about 1% to about 3% (w/w) cross-linked biocompatible polymer. In some embodiments, the hydrogel comprises about 1% to about 2.5% (w/w) cross-linked biocompatible polymer. In some embodiments, the hydrogel comprises about 1% to about 2% (w/w) cross-linked biocompatible polymer. In some embodiments, the hydrogel comprises about 2% to about 5% (w/w) cross-linked biocompatible polymer. In some embodiments, the hydrogel comprises about 2.5% to about 5% (w/w) cross-linked biocompatible polymer. In some embodiments, the hydrogel comprises about 2.5% to about 4% (w/w) cross-linked biocompatible polymer. In some embodiments, the hydrogel comprises about 2.5% to about 3% (w/w) cross-linked biocompatible polymer.

In some embodiments, the hydrogel comprises 1% to 10% (w/w) biocompatible polymer. In some embodiments, the hydrogel comprises 1% to 9% (w/w) biocompatible polymer. In some embodiments, the hydrogel comprises 1% to 8% (w/w) biocompatible polymer. In some embodiments, the hydrogel comprises 1% to 7.5% (w/w) biocompatible polymer. In some embodiments, the hydrogel comprises 1% to 7% (w/w) biocompatible polymer. In some embodiments, the hydrogel comprises 1% to 6% (w/w) biocompatible polymer. In some embodiments, the hydrogel comprises 1% to 5% (w/w) biocompatible polymer. In some embodiments, the hydrogel comprises 1% to 4% (w/w) biocompatible polymer. In some embodiments, the hydrogel comprises 1% to 3% (w/w) biocompatible polymer. In some embodiments, the hydrogel comprises 1% to 2.5% (w/w) biocompatible polymer. In some embodiments, the hydrogel comprises 1% to 2% (w/w) biocompatible polymer. In some embodiments, the hydrogel comprises 2% to 5% (w/w) biocompatible polymer. In some embodiments, the hydrogel comprises 2.5% to 5% (w/w) biocompatible polymer. In some embodiments, the hydrogel comprises 2.5% to 4% (w/w) biocompatible polymer. In some embodiments, the hydrogel comprises 2.5% to 3% (w/w) biocompatible polymer.

In some embodiments, the hydrogel comprises 1% to 10% (w/w) cross-linked biocompatible polymer. In some embodiments, the hydrogel comprises 1% to 9% (w/w) cross-linked biocompatible polymer. In some embodiments, the hydrogel comprises 1% to 8% (w/w) cross-linked biocompatible polymer. In some embodiments, the hydrogel comprises 1% to 7.5% (w/w) cross-linked biocompatible polymer. In some embodiments, the hydrogel comprises 1% to 7% (w/w) cross-linked biocompatible polymer. In some embodiments, the hydrogel comprises 1% to 6% (w/w) cross-linked biocompatible polymer. In some embodiments, the hydrogel comprises 1% to 5% (w/w) cross-linked biocompatible polymer. In some embodiments, the hydrogel comprises 1% to 4% (w/w) cross-linked biocompatible polymer. In some embodiments, the hydrogel comprises 1% to 3% (w/w) cross-linked biocompatible polymer. In some embodiments, the hydrogel comprises 1% to 2.5% (w/w) cross-linked biocompatible polymer. In some embodiments, the hydrogel comprises 1% to 2% (w/w) cross-linked biocompatible polymer. In some embodiments, the hydrogel comprises 2% to 5% (w/w) cross-linked biocompatible polymer. In some embodiments, the hydrogel comprises 2.5% to 5% (w/w) cross-linked biocompatible polymer. In some embodiments, the hydrogel comprises 2.5% to 4% (w/w) cross-linked biocompatible polymer. In some embodiments, the hydrogel comprises 2.5% to 3% (w/w) cross-linked biocompatible polymer.

In some embodiments, the hydrogel comprises about 1.0% (w/w) biocompatible polymer. In some embodiments, the hydrogel comprises about 1.1% (w/w) biocompatible polymer. In some embodiments, the hydrogel comprises about 1.2% (w/w) biocompatible polymer. In some embodiments, the hydrogel comprises about 1.3% (w/w) biocompatible polymer. In some embodiments, the hydrogel comprises about 1.4% (w/w) biocompatible polymer. In some embodiments, the hydrogel comprises about 1.5% (w/w) biocompatible polymer. In some embodiments, the hydrogel comprises about 1.6% (w/w) biocompatible polymer. In some embodiments, the hydrogel comprises about 1.7% (w/w) biocompatible polymer. In some embodiments, the hydrogel comprises about 1.8% (w/w) biocompatible polymer. In some embodiments, the hydrogel comprises about 1.9% (w/w) biocompatible polymer. In some embodiments, the hydrogel comprises about 2.0% (w/w) biocompatible polymer. In some embodiments, the hydrogel comprises about 2.1% (w/w) biocompatible polymer. In some embodiments, the hydrogel comprises about 2.2% (w/w) biocompatible polymer. In some embodiments, the hydrogel comprises about 2.3% (w/w) biocompatible polymer. In some embodiments, the hydrogel comprises about 2.4% (w/w) biocompatible polymer. In some embodiments, the hydrogel comprises about 2.5% (w/w) biocompatible polymer. In some embodiments, the hydrogel comprises about 2.6% (w/w) biocompatible polymer. In some embodiments, the hydrogel comprises about 2.7% (w/w) biocompatible polymer. In some embodiments, the hydrogel comprises about 2.8% (w/w) biocompatible polymer. In some embodiments, the hydrogel comprises about 2.9% (w/w) biocompatible polymer. In some embodiments, the hydrogel comprises about 3.0% (w/w) biocompatible polymer. In some embodiments, the hydrogel comprises about 3.1% (w/w) biocompatible polymer. In some embodiments, the hydrogel comprises about 3.2% (w/w) biocompatible polymer. In some embodiments, the hydrogel comprises about 3.3% (w/w) biocompatible polymer. In some embodiments, the hydrogel comprises about 3.4% (w/w) biocompatible polymer. In some embodiments, the hydrogel comprises about 3.5% (w/w) biocompatible polymer. In some embodiments, the hydrogel comprises about 3.6% (w/w) biocompatible polymer. In some embodiments, the hydrogel comprises about 3.7% (w/w) biocompatible polymer. In some embodiments, the hydrogel comprises about 3.8% (w/w) biocompatible polymer. In some embodiments, the hydrogel comprises about 3.9% (w/w) biocompatible polymer. In some embodiments, the hydrogel comprises about 4.0% (w/w) biocompatible polymer. In some embodiments, the hydrogel comprises about 4.1% (w/w) biocompatible polymer. In some embodiments, the hydrogel comprises about 4.2% (w/w) biocompatible polymer. In some embodiments, the hydrogel comprises about 4.3% (w/w) biocompatible polymer. In some embodiments, the hydrogel comprises about 4.4% (w/w) biocompatible polymer. In some embodiments, the hydrogel comprises about 4.5% (w/w) biocompatible polymer. In some embodiments, the hydrogel comprises about 4.6% (w/w) biocompatible polymer. In some embodiments, the hydrogel comprises about 4.7% (w/w) biocompatible polymer. In some embodiments, the hydrogel comprises about 4.8% (w/w) biocompatible polymer. In some embodiments, the hydrogel comprises about 4.9% (w/w) biocompatible polymer. In some embodiments, the hydrogel comprises about 5.0% (w/w) biocompatible polymer. In some embodiments, the hydrogel comprises about 5.5% (w/w) biocompatible polymer. In some embodiments, the hydrogel comprises about 6.0% (w/w) biocompatible polymer. In some embodiments, the hydrogel comprises about 6.5% (w/w) biocompatible polymer. In some embodiments, the hydrogel comprises about 7.0% (w/w) biocompatible polymer. In some embodiments, the hydrogel comprises about 7.5% (w/w) biocompatible polymer. In some embodiments, the hydrogel comprises about 8.0% (w/w) biocompatible polymer. In some embodiments, the hydrogel comprises about 8.5% (w/w) biocompatible polymer. In some embodiments, the hydrogel comprises about 9.0% (w/w) biocompatible polymer. In some embodiments, the hydrogel comprises about 9.5% (w/w) biocompatible polymer. In some embodiments, the hydrogel comprises about 10.0% (w/w) biocompatible polymer.

In some embodiments, the hydrogel comprises about 1.0% (w/w) cross-linked biocompatible polymer. In some embodiments, the hydrogel comprises about 1.1% (w/w) cross-linked biocompatible polymer. In some embodiments, the hydrogel comprises about 1.2% (w/w) cross-linked biocompatible polymer. In some embodiments, the hydrogel comprises about 1.3% (w/w) cross-linked biocompatible polymer. In some embodiments, the hydrogel comprises about 1.4% (w/w) cross-linked biocompatible polymer. In some embodiments, the hydrogel comprises about 1.5% (w/w) cross-linked biocompatible polymer. In some embodiments, the hydrogel comprises about 1.6% (w/w) cross-linked biocompatible polymer. In some embodiments, the hydrogel comprises about 1.7% (w/w) cross-linked biocompatible polymer. In some embodiments, the hydrogel comprises about 1.8% (w/w) cross-linked biocompatible polymer. In some embodiments, the hydrogel comprises about 1.9% (w/w) cross-linked biocompatible polymer. In some embodiments, the hydrogel comprises about 2.0% (w/w) cross-linked biocompatible polymer. In some embodiments, the hydrogel comprises about 2.1% (w/w) cross-linked biocompatible polymer. In some embodiments, the hydrogel comprises about 2.2% (w/w) cross-linked biocompatible polymer. In some embodiments, the hydrogel comprises about 2.3% (w/w) cross-linked biocompatible polymer. In some embodiments, the hydrogel comprises about 2.4% (w/w) cross-linked biocompatible polymer. In some embodiments, the hydrogel comprises about 2.5% (w/w) cross-linked biocompatible polymer. In some embodiments, the hydrogel comprises about 2.6% (w/w) cross-linked biocompatible polymer. In some embodiments, the hydrogel comprises about 2.7% (w/w) cross-linked biocompatible polymer. In some embodiments, the hydrogel comprises about 2.8% (w/w) cross-linked biocompatible polymer. In some embodiments, the hydrogel comprises about 2.9% (w/w) cross-linked biocompatible polymer. In some embodiments, the hydrogel comprises about 3.0% (w/w) cross-linked biocompatible polymer. In some embodiments, the hydrogel comprises about 3.1% (w/w) cross-linked biocompatible polymer. In some embodiments, the hydrogel comprises about 3.2% (w/w) cross-linked biocompatible polymer. In some embodiments, the hydrogel comprises about 3.3% (w/w) cross-linked biocompatible polymer. In some embodiments, the hydrogel comprises about 3.4% (w/w) cross-linked biocompatible polymer. In some embodiments, the hydrogel comprises about 3.5% (w/w) cross-linked biocompatible polymer. In some embodiments, the hydrogel comprises about 3.6% (w/w) cross-linked biocompatible polymer. In some embodiments, the hydrogel comprises about 3.7% (w/w) cross-linked biocompatible polymer. In some embodiments, the hydrogel comprises about 3.8% (w/w) cross-linked biocompatible polymer. In some embodiments, the hydrogel comprises about 3.9% (w/w) cross-linked biocompatible polymer. In some embodiments, the hydrogel comprises about 4.0% (w/w) cross-linked biocompatible polymer. In some embodiments, the hydrogel comprises about 4.1% (w/w) cross-linked biocompatible polymer. In some embodiments, the hydrogel comprises about 4.2% (w/w) cross-linked biocompatible polymer. In some embodiments, the hydrogel comprises about 4.3% (w/w) cross-linked biocompatible polymer. In some embodiments, the hydrogel comprises about 4.4% (w/w) cross-linked biocompatible polymer. In some embodiments, the hydrogel comprises about 4.5% (w/w) cross-linked biocompatible polymer. In some embodiments, the hydrogel comprises about 4.6% (w/w) cross-linked biocompatible polymer. In some embodiments, the hydrogel comprises about 4.7% (w/w) cross-linked biocompatible polymer. In some embodiments, the hydrogel comprises about 4.8% (w/w) cross-linked biocompatible polymer. In some embodiments, the hydrogel comprises about 4.9% (w/w) cross-linked biocompatible polymer. In some embodiments, the hydrogel comprises about 5.0% (w/w) cross-linked biocompatible polymer. In some embodiments, the hydrogel comprises about 5.5% (w/w) cross-linked biocompatible polymer. In some embodiments, the hydrogel comprises about 6.0% (w/w) cross-linked biocompatible polymer. In some embodiments, the hydrogel comprises about 6.5% (w/w) cross-linked biocompatible polymer. In some embodiments, the hydrogel comprises about 7.0% (w/w) cross-linked biocompatible polymer. In some embodiments, the hydrogel comprises about 7.5% (w/w) cross-linked biocompatible polymer. In some embodiments, the hydrogel comprises about 8.0% (w/w) cross-linked biocompatible polymer. In some embodiments, the hydrogel comprises about 8.5% (w/w) cross-linked biocompatible polymer. In some embodiments, the hydrogel comprises about 9.0% (w/w) cross-linked biocompatible polymer. In some embodiments, the hydrogel comprises about 9.5% (w/w) cross-linked biocompatible polymer. In some embodiments, the hydrogel comprises about 10.0% (w/w) cross-linked biocompatible polymer.

In some embodiments, the hydrogel comprises 1.0% (w/w) biocompatible polymer. In some embodiments, the hydrogel comprises 1.1% (w/w) biocompatible polymer. In some embodiments, the hydrogel comprises 1.2% (w/w) biocompatible polymer. In some embodiments, the hydrogel comprises 1.3% (w/w) biocompatible polymer. In some embodiments, the hydrogel comprises 1.4% (w/w) biocompatible polymer. In some embodiments, the hydrogel comprises 1.5% (w/w) biocompatible polymer. In some embodiments, the hydrogel comprises 1.6% (w/w) biocompatible polymer. In some embodiments, the hydrogel comprises 1.7% (w/w) biocompatible polymer. In some embodiments, the hydrogel comprises 1.8% (w/w) biocompatible polymer. In some embodiments, the hydrogel comprises 1.9% (w/w) biocompatible polymer. In some embodiments, the hydrogel comprises 2.0% (w/w) biocompatible polymer. In some embodiments, the hydrogel comprises 2.1% (w/w) biocompatible polymer. In some embodiments, the hydrogel comprises 2.2% (w/w) biocompatible polymer. In some embodiments, the hydrogel comprises 2.3% (w/w) biocompatible polymer. In some embodiments, the hydrogel comprises 2.4% (w/w) biocompatible polymer. In some embodiments, the hydrogel comprises 2.5% (w/w) biocompatible polymer. In some embodiments, the hydrogel comprises 2.6% (w/w) biocompatible polymer. In some embodiments, the hydrogel comprises 2.7% (w/w) biocompatible polymer. In some embodiments, the hydrogel comprises 2.8% (w/w) biocompatible polymer. In some embodiments, the hydrogel comprises 2.9% (w/w) biocompatible polymer. In some embodiments, the hydrogel comprises 3.0% (w/w) biocompatible polymer. In some embodiments, the hydrogel comprises 3.1% (w/w) biocompatible polymer. In some embodiments, the hydrogel comprises 3.2% (w/w) biocompatible polymer. In some embodiments, the hydrogel comprises 3.3% (w/w) biocompatible polymer. In some embodiments, the hydrogel comprises 3.4% (w/w) biocompatible polymer. In some embodiments, the hydrogel comprises 3.5% (w/w) biocompatible polymer. In some embodiments, the hydrogel comprises 3.6% (w/w) biocompatible polymer. In some embodiments, the hydrogel comprises 3.7% (w/w) biocompatible polymer. In some embodiments, the hydrogel comprises 3.8% (w/w) biocompatible polymer. In some embodiments, the hydrogel comprises 3.9% (w/w) biocompatible polymer. In some embodiments, the hydrogel comprises 4.0% (w/w) biocompatible polymer. In some embodiments, the hydrogel comprises 4.1% (w/w) biocompatible polymer. In some embodiments, the hydrogel comprises 4.2% (w/w) biocompatible polymer. In some embodiments, the hydrogel comprises 4.3% (w/w) biocompatible polymer. In some embodiments, the hydrogel comprises 4.4% (w/w) biocompatible polymer. In some embodiments, the hydrogel comprises 4.5% (w/w) biocompatible polymer. In some embodiments, the hydrogel comprises 4.6% (w/w) biocompatible polymer. In some embodiments, the hydrogel comprises 4.7% (w/w) biocompatible polymer. In some embodiments, the hydrogel comprises 4.8% (w/w) biocompatible polymer. In some embodiments, the hydrogel comprises 4.9% (w/w) biocompatible polymer. In some embodiments, the hydrogel comprises 5.0% (w/w) biocompatible polymer. In some embodiments, the hydrogel comprises 5.5% (w/w) biocompatible polymer. In some embodiments, the hydrogel comprises 6.0% (w/w) biocompatible polymer. In some embodiments, the hydrogel comprises 6.5% (w/w) biocompatible polymer. In some embodiments, the hydrogel comprises 7.0% (w/w) biocompatible polymer. In some embodiments, the hydrogel comprises 7.5% (w/w) biocompatible polymer. In some embodiments, the hydrogel comprises 8.0% (w/w) biocompatible polymer. In some embodiments, the hydrogel comprises 8.5% (w/w) biocompatible polymer. In some embodiments, the hydrogel comprises 9.0% (w/w) biocompatible polymer. In some embodiments, the hydrogel comprises 9.5% (w/w) biocompatible polymer. In some embodiments, the hydrogel comprises 10.0% (w/w) biocompatible polymer.

In some embodiments, the hydrogel comprises 1.0% (w/w) cross-linked biocompatible polymer. In some embodiments, the hydrogel comprises 1.1% (w/w) cross-linked biocompatible polymer. In some embodiments, the hydrogel comprises 1.2% (w/w) cross-linked biocompatible polymer. In some embodiments, the hydrogel comprises 1.3% (w/w) cross-linked biocompatible polymer. In some embodiments, the hydrogel comprises 1.4% (w/w) cross-linked biocompatible polymer. In some embodiments, the hydrogel comprises 1.5% (w/w) cross-linked biocompatible polymer. In some embodiments, the hydrogel comprises 1.6% (w/w) cross-linked biocompatible polymer. In some embodiments, the hydrogel comprises 1.7% (w/w) cross-linked biocompatible polymer. In some embodiments, the hydrogel comprises 1.8% (w/w) cross-linked biocompatible polymer. In some embodiments, the hydrogel comprises 1.9% (w/w) cross-linked biocompatible polymer. In some embodiments, the hydrogel comprises 2.0% (w/w) cross-linked biocompatible polymer. In some embodiments, the hydrogel comprises 2.1% (w/w) cross-linked biocompatible polymer. In some embodiments, the hydrogel comprises 2.2% (w/w) cross-linked biocompatible polymer. In some embodiments, the hydrogel comprises 2.3% (w/w) cross-linked biocompatible polymer. In some embodiments, the hydrogel comprises 2.4% (w/w) cross-linked biocompatible polymer. In some embodiments, the hydrogel comprises 2.5% (w/w) cross-linked biocompatible polymer. In some embodiments, the hydrogel comprises 2.6% (w/w) cross-linked biocompatible polymer. In some embodiments, the hydrogel comprises 2.7% (w/w) cross-linked biocompatible polymer. In some embodiments, the hydrogel comprises 2.8% (w/w) cross-linked biocompatible polymer. In some embodiments, the hydrogel comprises 2.9% (w/w) cross-linked biocompatible polymer. In some embodiments, the hydrogel comprises 3.0% (w/w) cross-linked biocompatible polymer. In some embodiments, the hydrogel comprises 3.1% (w/w) cross-linked biocompatible polymer. In some embodiments, the hydrogel comprises 3.2% (w/w) cross-linked biocompatible polymer. In some embodiments, the hydrogel comprises 3.3% (w/w) cross-linked biocompatible polymer. In some embodiments, the hydrogel comprises 3.4% (w/w) cross-linked biocompatible polymer. In some embodiments, the hydrogel comprises 3.5% (w/w) cross-linked biocompatible polymer. In some embodiments, the hydrogel comprises 3.6% (w/w) cross-linked biocompatible polymer. In some embodiments, the hydrogel comprises 3.7% (w/w) cross-linked biocompatible polymer. In some embodiments, the hydrogel comprises 3.8% (w/w) cross-linked biocompatible polymer. In some embodiments, the hydrogel comprises 3.9% (w/w) cross-linked biocompatible polymer. In some embodiments, the hydrogel comprises 4.0% (w/w) cross-linked biocompatible polymer. In some embodiments, the hydrogel comprises 4.1% (w/w) cross-linked biocompatible polymer. In some embodiments, the hydrogel comprises 4.2% (w/w) cross-linked biocompatible polymer. In some embodiments, the hydrogel comprises 4.3% (w/w) cross-linked biocompatible polymer. In some embodiments, the hydrogel comprises 4.4% (w/w) cross-linked biocompatible polymer. In some embodiments, the hydrogel comprises 4.5% (w/w) cross-linked biocompatible polymer. In some embodiments, the hydrogel comprises 4.6% (w/w) cross-linked biocompatible polymer. In some embodiments, the hydrogel comprises 4.7% (w/w) cross-linked biocompatible polymer. In some embodiments, the hydrogel comprises 4.8% (w/w) cross-linked biocompatible polymer. In some embodiments, the hydrogel comprises 4.9% (w/w) cross-linked biocompatible polymer. In some embodiments, the hydrogel comprises 5.0% (w/w) cross-linked biocompatible polymer. In some embodiments, the hydrogel comprises 5.5% (w/w) cross-linked biocompatible polymer. In some embodiments, the hydrogel comprises 6.0% (w/w) cross-linked biocompatible polymer. In some embodiments, the hydrogel comprises 6.5% (w/w) cross-linked biocompatible polymer. In some embodiments, the hydrogel comprises 7.0% (w/w) cross-linked biocompatible polymer. In some embodiments, the hydrogel comprises 7.5% (w/w) cross-linked biocompatible polymer. In some embodiments, the hydrogel comprises 8.0% (w/w) cross-linked biocompatible polymer. In some embodiments, the hydrogel comprises 8.5% (w/w) cross-linked biocompatible polymer. In some embodiments, the hydrogel comprises 9.0% (w/w) cross-linked biocompatible polymer. In some embodiments, the hydrogel comprises 9.5% (w/w) cross-linked biocompatible polymer. In some embodiments, the hydrogel comprises 10.0% (w/w) cross-linked biocompatible polymer.

In a preferred embodiment, the bulking agent is a polyacrylamide hydrogel (PAHG). In some embodiments, the PAHG comprises 1% to 10% (w/w) polyacrylamide. In some embodiments, the PAHG comprises 1% to 9% (w/w) polyacrylamide. In some embodiments, the PAHG comprises 1% to 8% (w/w) polyacrylamide. In some embodiments, the PAHG comprises 1% to 7.5% (w/w) polyacrylamide. In some embodiments, the PAHG comprises 1% to 7% (w/w) polyacrylamide. In some embodiments, the PAHG comprises 1% to 6% (w/w) polyacrylamide. In some embodiments, the PAHG comprises 1% to 5% (w/w) polyacrylamide. In some embodiments, the PAHG comprises 1% to 4% (w/w) polyacrylamide. In some embodiments, the PAHG comprises 1% to 3% (w/w) polyacrylamide. In some embodiments, the PAHG comprises 1% to 2.5% (w/w) polyacrylamide. In some embodiments, the PAHG comprises 1% to 2% (w/w) polyacrylamide. In some embodiments, the PAHG comprises 2% to 5% (w/w) polyacrylamide. In some embodiments, the PAHG comprises 2.5% to 5% (w/w) polyacrylamide. In some embodiments, the PAHG comprises 2.5% to 4% (w/w) polyacrylamide. In some embodiments, the PAHG comprises 2.5% to 3% (w/w) polyacrylamide.

In some embodiments, the PAHG comprises 1% to 10% (w/w) cross-linked polyacrylamide. In some embodiments, the PAHG comprises 1% to 9% (w/w) cross-linked polyacrylamide. In some embodiments, the PAHG comprises 1% to 8% (w/w) cross-linked polyacrylamide. In some embodiments, the PAHG comprises 1% to 7.5% (w/w) cross-linked polyacrylamide. In some embodiments, the PAHG comprises 1% to 7% (w/w) cross-linked polyacrylamide. In some embodiments, the PAHG comprises 1% to 6% (w/w) cross-linked polyacrylamide. In some embodiments, the PAHG comprises 1% to 5% (w/w) cross-linked polyacrylamide. In some embodiments, the PAHG comprises 1% to 4% (w/w) cross-linked polyacrylamide. In some embodiments, the PAHG comprises 1% to 3% (w/w) cross-linked polyacrylamide. In some embodiments, the PAHG comprises 1% to 2.5% (w/w) cross-linked polyacrylamide. In some embodiments, the PAHG comprises 1% to 2% (w/w) cross-linked polyacrylamide. In some embodiments, the PAHG comprises 2% to 5% (w/w) cross-linked polyacrylamide. In some embodiments, the PAHG comprises 2.5% to 5% (w/w) cross-linked polyacrylamide. In some embodiments, the PAHG comprises 2.5% to 4% (w/w) cross-linked polyacrylamide. In some embodiments, the PAHG comprises 2.5% to 3% (w/w) cross-linked polyacrylamide.

In some embodiments, the PAHG comprises about 1.0% (w/w) polyacrylamide. In some embodiments, the PAHG comprises about 1.1% (w/w) polyacrylamide. In some embodiments, the PAHG comprises about 1.2% (w/w) polyacrylamide. In some embodiments, the PAHG comprises about 1.3% (w/w) polyacrylamide. In some embodiments, the PAHG comprises about 1.4% (w/w) polyacrylamide. In some embodiments, the PAHG comprises about 1.5% (w/w) polyacrylamide. In some embodiments, the PAHG comprises about 1.6% (w/w) polyacrylamide. In some embodiments, the PAHG comprises about 1.7% (w/w) polyacrylamide. In some embodiments, the PAHG comprises about 1.8% (w/w) polyacrylamide. In some embodiments, the PAHG comprises about 1.9% (w/w) polyacrylamide. In some embodiments, the PAHG comprises about 2.0% (w/w) polyacrylamide. In some embodiments, the PAHG comprises about 2.1% (w/w) polyacrylamide. In some embodiments, the PAHG comprises about 2.2% (w/w) polyacrylamide. In some embodiments, the PAHG comprises about 2.3% (w/w) polyacrylamide. In some embodiments, the PAHG comprises about 2.4% (w/w) polyacrylamide. In some embodiments, the PAHG comprises about 2.5% (w/w) polyacrylamide. In some embodiments, the PAHG comprises about 2.6% (w/w) polyacrylamide. In some embodiments, the PAHG comprises about 2.7% (w/w) polyacrylamide. In some embodiments, the PAHG comprises about 2.8% (w/w) polyacrylamide. In some embodiments, the PAHG comprises about 2.9% (w/w) polyacrylamide. In some embodiments, the PAHG comprises about 3.0% (w/w) polyacrylamide. In some embodiments, the PAHG comprises about 3.1% (w/w) polyacrylamide. In some embodiments, the PAHG comprises about 3.2% (w/w) polyacrylamide. In some embodiments, the PAHG comprises about 3.3% (w/w) polyacrylamide. In some embodiments, the PAHG comprises about 3.4% (w/w) polyacrylamide. In some embodiments, the PAHG comprises about 3.5% (w/w) polyacrylamide. In some embodiments, the PAHG comprises about 3.6% (w/w) polyacrylamide. In some embodiments, the PAHG comprises about 3.7% (w/w) polyacrylamide. In some embodiments, the PAHG comprises about 3.8% (w/w) polyacrylamide. In some embodiments, the PAHG comprises about 3.9% (w/w) polyacrylamide. In some embodiments, the PAHG comprises about 4.0% (w/w) polyacrylamide. In some embodiments, the PAHG comprises about 4.1% (w/w) polyacrylamide. In some embodiments, the PAHG comprises about 4.2% (w/w) polyacrylamide. In some embodiments, the PAHG comprises about 4.3% (w/w) polyacrylamide. In some embodiments, the PAHG comprises about 4.4% (w/w) polyacrylamide. In some embodiments, the PAHG comprises about 4.5% (w/w) polyacrylamide. In some embodiments, the PAHG comprises about 4.6% (w/w) polyacrylamide. In some embodiments, the PAHG comprises about 4.7% (w/w) polyacrylamide. In some embodiments, the PAHG comprises about 4.8% (w/w) polyacrylamide. In some embodiments, the PAHG comprises about 4.9% (w/w) polyacrylamide. In some embodiments, the PAHG comprises about 5.0% (w/w) polyacrylamide. In some embodiments, the PAHG comprises about 5.5% (w/w) polyacrylamide. In some embodiments, the PAHG comprises about 6.0% (w/w) polyacrylamide. In some embodiments, the PAHG comprises about 6.5% (w/w) polyacrylamide. In some embodiments, the PAHG comprises about 7.0% (w/w) polyacrylamide. In some embodiments, the PAHG comprises about 7.5% (w/w) polyacrylamide. In some embodiments, the PAHG comprises about 8.0% (w/w) polyacrylamide. In some embodiments, the PAHG comprises about 8.5% (w/w) polyacrylamide. In some embodiments, the PAHG comprises about 9.0% (w/w) polyacrylamide. In some embodiments, the PAHG comprises about 9.5% (w/w) polyacrylamide. In some embodiments, the PAHG comprises about 10.0% (w/w) polyacrylamide.

In some embodiments, the PAHG comprises about 1.0% (w/w) cross-linked polyacrylamide. In some embodiments, the PAHG comprises about 1.1% (w/w) cross-linked polyacrylamide. In some embodiments, the PAHG comprises about 1.2% (w/w) cross-linked polyacrylamide. In some embodiments, the PAHG comprises about 1.3% (w/w) cross-linked polyacrylamide. In some embodiments, the PAHG comprises about 1.4% (w/w) cross-linked polyacrylamide. In some embodiments, the PAHG comprises about 1.5% (w/w) cross-linked polyacrylamide. In some embodiments, the PAHG comprises about 1.6% (w/w) cross-linked polyacrylamide. In some embodiments, the PAHG comprises about 1.7% (w/w) cross-linked polyacrylamide. In some embodiments, the PAHG comprises about 1.8% (w/w) cross-linked polyacrylamide. In some embodiments, the PAHG comprises about 1.9% (w/w) cross-linked polyacrylamide. In some embodiments, the PAHG comprises about 2.0% (w/w) cross-linked polyacrylamide. In some embodiments, the PAHG comprises about 2.1% (w/w) cross-linked polyacrylamide. In some embodiments, the PAHG comprises about 2.2% (w/w) cross-linked polyacrylamide. In some embodiments, the PAHG comprises about 2.3% (w/w) cross-linked polyacrylamide. In some embodiments, the PAHG comprises about 2.4% (w/w) cross-linked polyacrylamide. In some embodiments, the PAHG comprises about 2.5% (w/w) cross-linked polyacrylamide. In some embodiments, the PAHG comprises about 2.6% (w/w) cross-linked polyacrylamide. In some embodiments, the PAHG comprises about 2.7% (w/w) cross-linked polyacrylamide. In some embodiments, the PAHG comprises about 2.8% (w/w) cross-linked polyacrylamide. In some embodiments, the PAHG comprises about 2.9% (w/w) cross-linked polyacrylamide. In some embodiments, the PAHG comprises about 3.0% (w/w) cross-linked polyacrylamide. In some embodiments, the PAHG comprises about 3.1% (w/w) cross-linked polyacrylamide. In some embodiments, the PAHG comprises about 3.2% (w/w) cross-linked polyacrylamide. In some embodiments, the PAHG comprises about 3.3% (w/w) cross-linked polyacrylamide. In some embodiments, the PAHG comprises about 3.4% (w/w) cross-linked polyacrylamide. In some embodiments, the PAHG comprises about 3.5% (w/w) cross-linked polyacrylamide. In some embodiments, the PAHG comprises about 3.6% (w/w) cross-linked polyacrylamide. In some embodiments, the PAHG comprises about 3.7% (w/w) cross-linked polyacrylamide. In some embodiments, the PAHG comprises about 3.8% (w/w) cross-linked polyacrylamide. In some embodiments, the PAHG comprises about 3.9% (w/w) cross-linked polyacrylamide. In some embodiments, the PAHG comprises about 4.0% (w/w) cross-linked polyacrylamide. In some embodiments, the PAHG comprises about 4.1% (w/w) cross-linked polyacrylamide. In some embodiments, the PAHG comprises about 4.2% (w/w) cross-linked polyacrylamide. In some embodiments, the PAHG comprises about 4.3% (w/w) cross-linked polyacrylamide. In some embodiments, the PAHG comprises about 4.4% (w/w) cross-linked polyacrylamide. In some embodiments, the PAHG comprises about 4.5% (w/w) cross-linked polyacrylamide. In some embodiments, the PAHG comprises about 4.6% (w/w) cross-linked polyacrylamide. In some embodiments, the PAHG comprises about 4.7% (w/w) cross-linked polyacrylamide. In some embodiments, the PAHG comprises about 4.8% (w/w) cross-linked polyacrylamide. In some embodiments, the PAHG comprises about 4.9% (w/w) cross-linked polyacrylamide. In some embodiments, the PAHG comprises about 5.0% (w/w) cross-linked polyacrylamide. In some embodiments, the PAHG comprises about 5.5% (w/w) cross-linked polyacrylamide. In some embodiments, the PAHG comprises about 6.0% (w/w) cross-linked polyacrylamide. In some embodiments, the PAHG comprises about 6.5% (w/w) cross-linked polyacrylamide. In some embodiments, the PAHG comprises about 7.0% (w/w) cross-linked polyacrylamide. In some embodiments, the PAHG comprises about 7.5% (w/w) cross-linked polyacrylamide. In some embodiments, the PAHG comprises about 8.0% (w/w) cross-linked polyacrylamide. In some embodiments, the PAHG comprises about 8.5% (w/w) cross-linked polyacrylamide. In some embodiments, the PAHG comprises about 9.0% (w/w) cross-linked polyacrylamide. In some embodiments, the PAHG comprises about 9.5% (w/w) cross-linked polyacrylamide. In some embodiments, the PAHG comprises about 10.0% (w/w) cross-linked polyacrylamide.

In some embodiments, the PAHG comprises 1.0% (w/w) polyacrylamide. In some embodiments, the PAHG comprises 1.1% (w/w) polyacrylamide. In some embodiments, the PAHG comprises 1.2% (w/w) polyacrylamide. In some embodiments, the PAHG comprises 1.3% (w/w) polyacrylamide. In some embodiments, the PAHG comprises 1.4% (w/w) polyacrylamide. In some embodiments, the PAHG comprises 1.5% (w/w) polyacrylamide. In some embodiments, the PAHG comprises 1.6% (w/w) polyacrylamide. In some embodiments, the PAHG comprises 1.7% (w/w) polyacrylamide. In some embodiments, the PAHG comprises 1.8% (w/w) polyacrylamide. In some embodiments, the PAHG comprises 1.9% (w/w) polyacrylamide. In some embodiments, the PAHG comprises 2.0% (w/w) polyacrylamide. In some embodiments, the PAHG comprises 2.1% (w/w) polyacrylamide. In some embodiments, the PAHG comprises 2.2% (w/w) polyacrylamide. In some embodiments, the PAHG comprises 2.3% (w/w) polyacrylamide. In some embodiments, the PAHG comprises 2.4% (w/w) polyacrylamide. In some embodiments, the PAHG comprises 2.5% (w/w) polyacrylamide. In some embodiments, the PAHG comprises 2.6% (w/w) polyacrylamide. In some embodiments, the PAHG comprises 2.7% (w/w) polyacrylamide. In some embodiments, the PAHG comprises 2.8% (w/w) polyacrylamide. In some embodiments, the PAHG comprises 2.9% (w/w) polyacrylamide. In some embodiments, the PAHG comprises 3.0% (w/w) polyacrylamide. In some embodiments, the PAHG comprises 3.1% (w/w) polyacrylamide. In some embodiments, the PAHG comprises 3.2% (w/w) polyacrylamide. In some embodiments, the PAHG comprises 3.3% (w/w) polyacrylamide. In some embodiments, the PAHG comprises 3.4% (w/w) polyacrylamide. In some embodiments, the PAHG comprises 3.5% (w/w) polyacrylamide. In some embodiments, the PAHG comprises 3.6% (w/w) polyacrylamide. In some embodiments, the PAHG comprises 3.7% (w/w) polyacrylamide. In some embodiments, the PAHG comprises 3.8% (w/w) polyacrylamide. In some embodiments, the PAHG comprises 3.9% (w/w) polyacrylamide. In some embodiments, the PAHG comprises 4.0% (w/w) polyacrylamide. In some embodiments, the PAHG comprises 4.1% (w/w) polyacrylamide. In some embodiments, the PAHG comprises 4.2% (w/w) polyacrylamide. In some embodiments, the PAHG comprises 4.3% (w/w) polyacrylamide. In some embodiments, the PAHG comprises 4.4% (w/w) polyacrylamide. In some embodiments, the PAHG comprises 4.5% (w/w) polyacrylamide. In some embodiments, the PAHG comprises 4.6% (w/w) polyacrylamide. In some embodiments, the PAHG comprises 4.7% (w/w) polyacrylamide. In some embodiments, the PAHG comprises 4.8% (w/w) polyacrylamide. In some embodiments, the PAHG comprises 4.9% (w/w) polyacrylamide. In some embodiments, the PAHG comprises 5.0% (w/w) polyacrylamide. In some embodiments, the PAHG comprises 5.5% (w/w) polyacrylamide. In some embodiments, the PAHG comprises 6.0% (w/w) polyacrylamide. In some embodiments, the PAHG comprises 6.5% (w/w) polyacrylamide. In some embodiments, the PAHG comprises 7.0% (w/w) polyacrylamide. In some embodiments, the PAHG comprises 7.5% (w/w) polyacrylamide. In some embodiments, the PAHG comprises 8.0% (w/w) polyacrylamide. In some embodiments, the PAHG comprises 8.5% (w/w) polyacrylamide. In some embodiments, the PAHG comprises 9.0% (w/w) polyacrylamide. In some embodiments, the PAHG comprises 9.5% (w/w) polyacrylamide. In some embodiments, the PAHG comprises 10.0% (w/w) polyacrylamide.

In some embodiments, the PAHG comprises 1.0% (w/w) cross-linked polyacrylamide. In some embodiments, the PAHG comprises 1.1% (w/w) cross-linked polyacrylamide. In some embodiments, the PAHG comprises 1.2% (w/w) cross-linked polyacrylamide. In some embodiments, the PAHG comprises 1.3% (w/w) cross-linked polyacrylamide. In some embodiments, the PAHG comprises 1.4% (w/w) cross-linked polyacrylamide. In some embodiments, the PAHG comprises 1.5% (w/w) cross-linked polyacrylamide. In some embodiments, the PAHG comprises 1.6% (w/w) cross-linked polyacrylamide. In some embodiments, the PAHG comprises 1.7% (w/w) cross-linked polyacrylamide. In some embodiments, the PAHG comprises 1.8% (w/w) cross-linked polyacrylamide. In some embodiments, the PAHG comprises 1.9% (w/w) cross-linked polyacrylamide. In some embodiments, the PAHG comprises 2.0% (w/w) cross-linked polyacrylamide. In some embodiments, the PAHG comprises 2.1% (w/w) cross-linked polyacrylamide. In some embodiments, the PAHG comprises 2.2% (w/w) cross-linked polyacrylamide. In some embodiments, the PAHG comprises 2.3% (w/w) cross-linked polyacrylamide. In some embodiments, the PAHG comprises 2.4% (w/w) cross-linked polyacrylamide. In some embodiments, the PAHG comprises 2.5% (w/w) cross-linked polyacrylamide. In some embodiments, the PAHG comprises 2.6% (w/w) cross-linked polyacrylamide. In some embodiments, the PAHG comprises 2.7% (w/w) cross-linked polyacrylamide. In some embodiments, the PAHG comprises 2.8% (w/w) cross-linked polyacrylamide. In some embodiments, the PAHG comprises 2.9% (w/w) cross-linked polyacrylamide. In some embodiments, the PAHG comprises 3.0% (w/w) cross-linked polyacrylamide. In some embodiments, the PAHG comprises 3.1% (w/w) cross-linked polyacrylamide. In some embodiments, the PAHG comprises 3.2% (w/w) cross-linked polyacrylamide. In some embodiments, the PAHG comprises 3.3% (w/w) cross-linked polyacrylamide. In some embodiments, the PAHG comprises 3.4% (w/w) cross-linked polyacrylamide. In some embodiments, the PAHG comprises 3.5% (w/w) cross-linked polyacrylamide. In some embodiments, the PAHG comprises 3.6% (w/w) cross-linked polyacrylamide. In some embodiments, the PAHG comprises 3.7% (w/w) cross-linked polyacrylamide. In some embodiments, the PAHG comprises 3.8% (w/w) cross-linked polyacrylamide. In some embodiments, the PAHG comprises 3.9% (w/w) cross-linked polyacrylamide. In some embodiments, the PAHG comprises 4.0% (w/w) cross-linked polyacrylamide. In some embodiments, the PAHG comprises 4.1% (w/w) cross-linked polyacrylamide. In some embodiments, the PAHG comprises 4.2% (w/w) cross-linked polyacrylamide. In some embodiments, the PAHG comprises 4.3% (w/w) cross-linked polyacrylamide. In some embodiments, the PAHG comprises 4.4% (w/w) cross-linked polyacrylamide. In some embodiments, the PAHG comprises 4.5% (w/w) cross-linked polyacrylamide. In some embodiments, the PAHG comprises 4.6% (w/w) cross-linked polyacrylamide. In some embodiments, the PAHG comprises 4.7% (w/w) cross-linked polyacrylamide. In some embodiments, the PAHG comprises 4.8% (w/w) cross-linked polyacrylamide. In some embodiments, the PAHG comprises 4.9% (w/w) cross-linked polyacrylamide. In some embodiments, the PAHG comprises 5.0% (w/w) cross-linked polyacrylamide. In some embodiments, the PAHG comprises 5.5% (w/w) cross-linked polyacrylamide. In some embodiments, the PAHG comprises 6.0% (w/w) cross-linked polyacrylamide. In some embodiments, the PAHG comprises 6.5% (w/w) cross-linked polyacrylamide. In some embodiments, the PAHG comprises 7.0% (w/w) cross-linked polyacrylamide. In some embodiments, the PAHG comprises 7.5% (w/w) cross-linked polyacrylamide. In some embodiments, the PAHG comprises 8.0% (w/w) cross-linked polyacrylamide. In some embodiments, the PAHG comprises 8.5% (w/w) cross-linked polyacrylamide. In some embodiments, the PAHG comprises 9.0% (w/w) cross-linked polyacrylamide. In some embodiments, the PAHG comprises 9.5% (w/w) cross-linked polyacrylamide. In some embodiments, the PAHG comprises 10.0% (w/w) cross-linked polyacrylamide.

In some embodiments, the method comprises administering multiple injections of bulking agent. In some embodiments, the method comprises one injection. In some embodiments, the method comprises two injections. In some embodiments, the method comprises three injections. In some embodiments, the method comprises four injections. In some embodiments, the method comprises five injections. In some embodiments, the method comprises six injections. In some embodiments, the method comprises seven injections. In some embodiments, the method comprises eight injections. In some embodiments, the method comprises nine injections. In some embodiments, the method comprises ten injections.

In some embodiments, the bulking agent (e.g., hydrogel) is delivered to the subject using the Bulkamid® System which is a kit containing two 1 ml prefilled syringes of bulking agent including for example PAHG (2.5% cross-linked polyacrylamide and 97.5% non-pyrogenic water (w/w)) with accompanying injection needles, a proprietary delivery system of a 4.5-inch zero-degree cystoscope designed specifically for the female urethra and a 5-inch rotatable sheath to facilitate the injection.

In some embodiments, about 1 ml to about 10 ml of bulking agent in administered into the external urethral sphincter of the subject. In some embodiments, about 2 to about 4 ml of bulking agent in administered into the external urethral sphincter of the subject. In some embodiments, about 1, 1.5, 2, 2.5, 3, 3.5, 4, 4.5, 5, 5.5, 6, 6.5, 7, 7.5, 8, 8.5, 9, 9.5, or 10 ml of bulking agent in administered into the external urethral sphincter of the subject. In some embodiments, 1, 1.5, 2, 2.5, 3, 3.5, 4, 4.5, 5, 5.5, 6, 6.5, 7, 7.5, 8, 8.5, 9, 9.5, or 10 ml of bulking agent in administered into the external urethral sphincter of the subject.

In some embodiments, the bulking agent is administered using pre-filled syringes. In some embodiments, each prefilled syringe comprises about 1 ml of bulking agent. In some embodiments, each prefilled syringe comprises 1 ml of bulking agent. In some embodiments, about 1 to about 10 pre-filled syringes of bulking agent are used to administer the bulking agent into the external urethral sphincter of the subject. In some embodiments, 1 to 10 pre-filled syringes of bulking agent are used to administer the bulking agent into the external urethral sphincter of the subject. In some embodiments, about 2 to about 4 pre-filled syringes of bulking agent are used to administer the bulking agent into the external urethral sphincter of the subject. In some embodiments, 2 to 4 pre-filled syringes of bulking agent are used to administer the bulking agent into the external urethral sphincter of the subject. In some embodiments, about 1, 2, 3, 4, 5, 6, 7, 8, 9, or 10 pre-filled syringes are used to administer the bulking agent into the external urethral sphincter of the subject. In some embodiments, 1, 2, 3, 4, 5, 6, 7, 8, 9, or 10 pre-filled syringes are used to administer the bulking agent into the external urethral sphincter of the subject.

In some embodiments, the bulking agent is substantially free of any other polymeric content. In some embodiments, the bulking agent further comprises pyrogen-free water, buffer or saline solution. In some embodiments, the bulking agent further comprises pyrogen-free water. In some embodiments, the bulking agent further comprises saline solution. In some embodiments, the bulking agent further comprises a buffer. Suitable saline solutions include but are not limited to the group selected from aqueous sodium chloride, Ringer's lactate solution, acetated Ringer's solution, a glucose solution, and dextrose in normal saline.

In some embodiments, the present disclosure provides educational instructions to the subject for improving the longevity of the CREST procedure. For example, in order to prevent spontaneous recurrence of SUI post procedure, the methods can include advising patients against typical pushing/straining behaviors while voiding. In some instances, providing such an instruction has mitigated against misalignment and/or hydrogel loss.

In some embodiments, the instructions include warning patients about pelvic muscle clenching and abdominal straining while voiding and/or defecating prior to injection of the PAHG. In addition, the instructions can include educating patients on practices including pelvic floor awareness, puborectalis release, diaphragmatic breathing during toileting, extensive bladder retraining, and the like allow patients to retain the hydrogel and remain dry.

### Pelvic Floor Awareness and Puborectalis Release

Many women demonstrate diminished sensorimotor control of their pelvic floor. Muscle, as demonstrated by inaccurate self-perception of their ability to engage their pelvic muscles to contract and relax. Accordingly, education related to pelvic floor dysfunction and methods for improving awareness and regaining control can improve the success of the procedure.

In some embodiments, the educational instructions include providing pelvic floor awareness education. The pelvic floor awareness can be initiated by the provider on the initial evaluation of any women presenting with lower urinary tract dysfunction regardless of the specific complaint. The pelvic floor awareness and puborectalis release techniques are taught to the subject through a physical pelvic exam. In some embodiments, the physical pelvic exam is performed by first properly positioning the subject. The proper position can include a supine recumbent position, on the back, not the buttocks. The feet are not in stirrups but instead the legs are positioned in a V with the soles of the feet on the table. In some embodiments, the pelvic exam includes providing the subject with an oral warning as the procedure can activate reactive pelvic floors with robust guarding reflexes that can spasm. In some embodiments, the pelvic exam can include performing a one-finger exam in order to assess the pathognomonic sign of a “tented up” puborectalis (PR) muscle. In some embodiments, the pelvic exam can include palpating the PR midline and 2 cm inward on the posterior wall of the vagina. In some embodiments, the pelvic exam can include asking the subject to perform a Kegel exercise which includes an enhanced clench (Super clench) including recruitment of abdominal muscles which can be confirmed by pressing on the abdomen of the subject. In some embodiments, the pelvic exam can include providing feedback that unwanted accessory muscles are being engaged. In some embodiments, in order to disengage accessory muscles, the exam includes instructing the subject to reverse the Kegel and relax. The instructions for reversing the Kegel can include asking the subject to take four deep breaths through the nose and out the mouth, while the physician applies gentle pressure in the middle of the PR at 12 o'clock while asking the subject to “let go”. In some embodiments, as the PR slowly releases tension, the vaginal opening elongates toward the table. In some embodiments, the pelvic exam can include asking the subject if she feels the “relax”. If a negative response is received, the procedure for reversing the Kegel can be repeated. In some embodiments, the pelvic exam can include confirming that a maximum pelvic relaxation or “ground zero” state has been achieved by palpating a separation of the right and left PR muscle in the midline as well as complete relaxation of the more superficial pelvic muscles. In some embodiments, the pelvic exam can include instructing the subject to remain in this relaxed position for approximately two minutes. In some embodiments, the pelvic exam can include retaining a finger in the introitus to ensure the subject does not re-clench the pelvic muscles. The educational instructions can include informing the subject that this is the position of pelvic relaxation is the one that should be obtained when urinating and defecting. The pelvic exam can then include removing the finger and asking the subject to continue holding the relaxation for as long as possible while being mindful of clenching behaviors throughout the day.

### Bladder Retraining

In some embodiments, the educational instructions can include retraining the bladder. In some embodiments, the bladder retaining can include instructing the subject to drink an average of 64 oz of mostly water. In some embodiments, the bladder retaining can include instructing the subject to void her bladder every two hours, preferably without encountering a significant urge to urinate, if possible. In some embodiments, the bladder retaining includes instructing the subject to use a proper toilet position. The proper toilet position can include leaning slightly forward and deep breathing to release the pelvic floor without pushing or straining. In some embodiments, the bladder retaining can include increasing the interval between voids by 15 minutes every week until the subject can void comfortably at a three-hour interval. In some embodiments, the bladder retaining results in a technique of pelvic floor release, that aids in improving toileting behaviors and preventing the progression of further problems related to SUI.

### Diaphragmatic Breathing

Parasympathetic stimulation has been shown to relax the levator muscles through nitrogenous release. Accordingly, embodiments of the educational instructions of the present disclosure include providing instructions for diaphragmatic breathing which is a common technique for stimulating parasympathetic relaxation. In some embodiments, instructing the use of diaphragmatic breathing is effective for releasing the levator muscles and opening the external sphincter, thereby facilitating unobstructed voiding. In some embodiments, the educational instructions are integral because clenching is an unconscious/reflexive behavior and simply alerting the woman to the nature of the problem and how it manifests in her life is enough to reverse the dysfunctional behavior. The education instructions can include instructions for deeply inhaling while engaging the diaphragm muscles and visualizing the uterine region filling with air. This combined physical and mental exercise has facilitated more effective and proper levator muscle relaxation.

### Kits

In some embodiments, the present disclosure provides kits containing one or more containers for dispensing the bulking agent as contemplated herein. The kit can include one or more injecting devices including one or more injection needles and/or syringes. The one or more injecting devices can be preloaded with an effective volume of bulking agent for effectively performing reconstruction of the external urinary sphincter. The preloaded volume can include a volume of up to 1 bulk dosage of the bulking agent. The preloaded volume can include 2 bulk dosages, 3 bulk dosages, 4 bulk dosages, 5 bulk dosages, 6 bulk dosages, 7 bulk dosages, 8 bulk dosages, 9 bulk dosages, 10 bulk dosages, or more than 10 bulk doses. For example, the kit can include two 1 ml prefilled syringes of bulking agent including for example PAHG (2.5% cross-linked polyacrylamide and 97.5% non-pyrogenic water (w/w)). In some embodiments, the kit can include a proprietary delivery system of a 4.5-inch zero-degree cystoscope designed specifically for the female urethra and a 5-inch rotatable sheath to facilitate the injection. Alternatively, the kit can include one or more containers loaded with a volume of bulking agent (e.g., PAHG) for drawing from to load the provided injecting devices. The one or more injecting devices can include one or more syringes, or the like. The kit can also include one or more instructions for using the bulking agent, instructions for loading the bulking agent into the one or more injecting devices, instructions for administering the bulking agent to a subject in need thereof, instructions for counseling patients in order to improve outcomes, or one or more combinations thereof. The contents of the kit can be contained in a packaging. The contents of the kit can be sterilized. The packaging can include sterile packaging. The kit can further include any instruments as understood in the art for use in administering the bulking agent to a subject including for example a cystoscope. The cystoscope can be sterile packaged individually or as part of the kit.

### EXAMPLES

1. Technique for Endoscopic Reconstruction of the External Urethral Sphincter with PAHG

In the past, there were concerns over injecting the external urethral sphincter (EUS) with a particulate bulking agent for fear of sphincter damage. One concern was that putting an inflammatory particulate substance into the EUS might run the risk of worsening sphincter function through a fibrotic process. Because the anatomy of the female urethra is generally poorly understood, even to practicing urologists, the EUS is a structure that is rarely visualized with routine cystourethroscopy. In some cases, it can be difficult to localize and there are no standard measurements to use. In general, it is anywhere from 2 to 3 cm distal to the bladder neck depending on the length of the urethra. Using this methodology, sphincter defects can be visualized and injected using Bulkamid®.

The details of the procedure are as follows:
1. After positioning the patient in lithotomy position, 2 mg of Versed (midazolam and 1 g of Ancef (cefazolin) is administered intravenously (IV).
2. 10 ml of 1% lidocaine is injected around the urethral opening and 2% viscous lidocaine is inserted into the meatus.
3. The Bulkamid® system is inserted and cystoscopy/urethroscopy is performed. By gently moving the scope/sheath back and forth over the posterior urethra, the urethral crest is identified. The procedure is done with high pressure irrigation. That is, the pressure bag is set at 150 cm H₂O to make sure seal can withstand this pressure level. The anatomy of the EUS (the urethrovaginal sphincter and compressor urethra) presents itself by using a high flow rate and the defects in the continuity of the sphincter muscle can be assessed. Generally speaking, the EUS is 3 cm distal to the bladder neck.
4. Next the rotatable sheath is turned to the 5 o'clock position and the needle is advanced directly into the inferior portion of the EUS so that the needle tip is ½ cm deep into the striated sphincter muscle. The bevel tip should be turned inward toward the urethral lumen. Approximately 0.5 ml to 1 ml of the hydrogel is injected depending on the nature of the defect so that a mound of tissue expansion pushes toward the midline.
5. Next the sheath is rotated clockwise to observe the divot between the base of the mound and the 7 o'clock position. The needle is inserted at this point and the right side of the urethrovaginal sphincter is injected with additional 0.5-1 ml of hydrogel (PAHG) as needed to create a continuous mound at the bottom of the EUS forming the bottom of the ring.
6. The sheath is turned to the 1 o'clock position and the defect in this area is assessed. Then, 0.5 ml of hydrogel is injected into the compressor urethra to create a mound in this region. The injection at the 11 o'clock position should complete the ring and the scope is pulled back to visually confirm the seal of the EUS
7. With the patient awake and the scope removed, with at least 250 ml saline left in the bladder, the patient is asked to cough. Cough training prior to procedure is useful and ensuring full wakefulness for cough to test repair is necessary. If there is any additional leakage noted with a strong cough, the scope is reinserted, and the defect was assessed. Injections in 0.5 ml increments are injected at the defect site and the cough test repeated. If no defect is seen, an additional injection of 0.5 ml at 6'oclock under the urethral crest can ensure a tighter seal.
8. The bladder is left halfway full (200 ml-250 ml) at the end of the case so voiding trial can be performed.

In some instances, the observed damage to the EUS has been severe, with parts of it completely missing. Once defects are located, the agent (Bulkamid®; 2.5% PAHG) is injected circumferentially causing a complete seal of the EUS without ever going near the bladder neck. This constitutes an endoscopic reconstruction of the EUS with PAHG. Aggressive cough testing is used to assess the success of the procedure. The results obtained have been far superior (approaching MUS). The top off (i.e., retreatment) rates are less and the retention rate (i.e., inability to urinate) is considerably less. No complications from the procedure have been observed.

In some instances, patient education related to the voiding dysfunction component of SUI significantly improves longevity of the CREST procedure. In order to prevent spontaneous recurrence of SUI post procedure, advising patients against typical pushing/straining behaviors while voiding has mitigated against misalignment and/or hydrogel loss.

Accordingly, all women are thoroughly cautioned about pelvic muscle clenching and abdominal straining while voiding and/or defecating prior to injection of the PAHG. In addition, educating patients on practices including pelvic floor awareness, puborectalis release, diaphragmatic breathing during toileting, extensive bladder retraining, and the like allow patients to retain the hydrogel and remain dry.

### 2. Cystoscopic Reconstruction/Realignment of External Sphincter Technique (CREST Procedure)

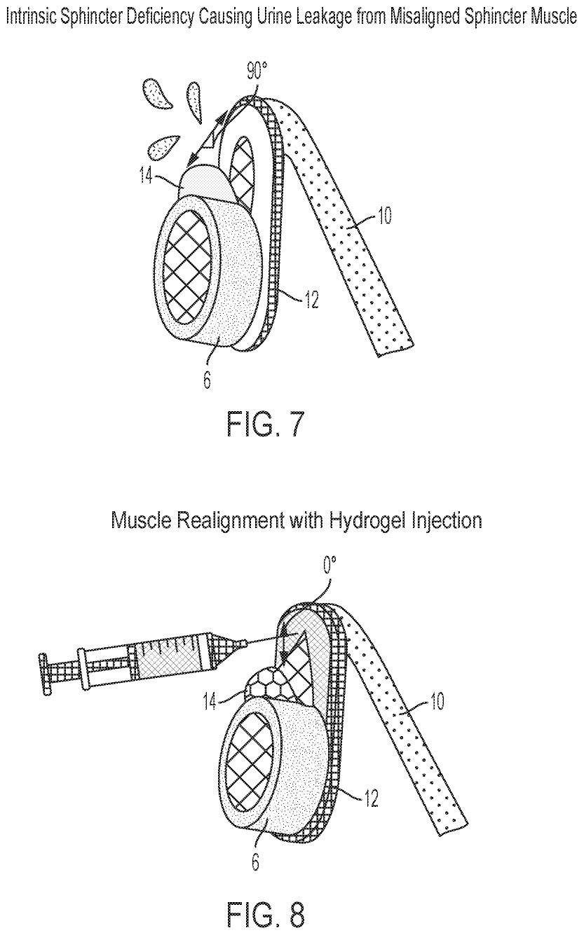

Urethral hypermobility causes the CU/UVS complex 10/12 to fail to make contact with the UC 14, due to a 90 degree or greater displacement of the anterior and posterior walls of the distal urethra, as shown in FIG. 7. A goal of the CREST procedure of present invention is to build up the structure of the urethral crest in volume and depth, as well as the volume of the CU/UVS complex 10/12, to facilitate a zero-degree displacement or aligned compression mechanism, as shown in FIG. 8. In patients without UH, the goal is not realignment but reconstruction and rebuilding of weakened sphincter tissues.

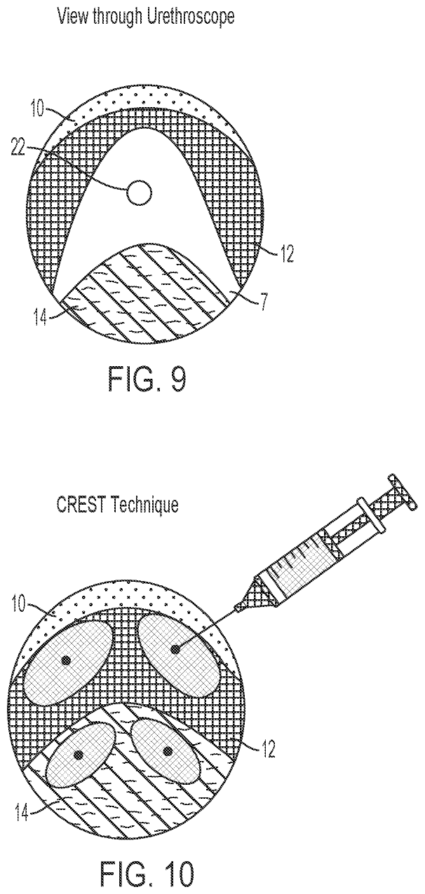

When entering the female urethra using an endoscopic video camera, such as a cystoscope with a zero-degree lens and a display screen, the structure first encountered is the UC 14 at the inferior part of the screen. By pulling the cystoscope back to the external meatus, the submucosal tissues can then be milked forward by placing a downward traction, which allows the UC 14 to come into view. In general, the UC 14 lies 1 cm or less from the external meatus. The location of the UC 14 in reference to the bladder neck 22 depends on the length of the urethra 8, and is on average, 3-4 cm distal. In order to visualize the CU/UVS complex 10/12 at the superior and lateral walls, a higher inflow of water may be used. This maneuver demonstrates visible defects in the closure mechanism and can help guide where to best perform the injection, shown in FIG. 9 which depicts a view of the urethra through a urethroscope.

FIG. 10 shows an injection of a bulking agent according to the CREST procedure. A goal of the injection is to create a visual seal between the UC 14 and the CU/UVS 10/12 that can be tested on the table by having the patient cough. In general, 3 ml of the bulking agent material is injected but this varies by patient anatomy and tissue properties. Because of the dynamic nature of the EUS, as compared to the IUS, there is little concern for over-injecting and causing urinary retention. If necessary, a second treatment can be performed after 4 to 6 weeks from the first procedure. The success rate of CREST so far approaches MUS at three months, with 65% requiring one treatment and 35% requiring two treatments. Moreover, there have been no observed complications from the procedure. The discovery of CREST and, consequently, a more complete understanding of the intricate components of the female continence mechanism is a breakthrough in the treatment of female stress urinary incontinence.

### 3. Clinical Data of the CREST Procedure

Between Jan. 21, 2022 and Oct. 28, 2022, 161 women having a mean age of 59 years old, who met the criteria for stress urinary incontinence or mixed urinary incontinence, (exclusion criteria of concomitant pelvic floor reconstruction, previous sling procedure, coexisting pelvic organ prolapse, neurogenic bladder or previous radiation therapy) underwent CREST. The procedures were all done by one surgeon in an ambulatory setting under mild sedation. The majority of women required 3 ml of Bulkamid® product.

Patient demographics are listed in Table 1 below. Following the procedure, 126 women were available for follow up. The majority of patients in this short-term study received 1 treatment (1.2 average). The median follow up was 38 days following the procedure. Of the participants, 113 patients were seen as a 3-month or greater follow up.

TABLE 1
Demographics

| | | All |
|---|---|---|
| | | participants |
| | | (n = 113) |
| | | Median f/u |
| | | ≥90 d |
| | All participants | (bulk 1/22- |
| | (n = 126) | 10/22) |
| Age | 59 (14.7) | 59 (14.6) |
| BMI (kg/m²) (mean, SD) | 29 (7) | 29 (6.5) |
| Parity (median, IQR) | 2 (1) | 2 (1) |
| Postmenopausal | 84 (67) | 75 (66) |
| Diabetes | 9 (7) | 7 (6) |
| Current smoker | 17 (13) | 16 (14) |
| Recurrent UTI | 20 (16) | 17 (15) |
| Stress urinary incontinence | 9 (7) | 8 (7) |
| Stress predominant mixed urinary | 21 (17) | 20 (18) |
| incontinence | | |
| Urgency predominant mixed urinary | 63 (50) | 29 (52) |
| incontinence | | |

Data are presented as n (%) unless otherwise noted.

Postoperative outcomes are listed in Table 2 below. Overall, 69% of women were objectively cured (cough stress test negative). Of the participants asked, 70% answered “yes” they were greater than 70% drier than before the injection and 86% stated that they no longer had leakage episodes.

TABLE 2
Postoperative Outcomes

| | All participants | All participants |
|---|---|---|
| | (n = 126) | (n = 113) |
| Subjective Improvement (Yes/No) | 108 (86) | 96 (85) |
| Subjective Cure ≥70% | 88 (70) | 78 (69) |
| Objective cure | 87 (69) | 76 (67) |
| Mean number of treatments (SD) | 1.2 (0.4) | 1.3 (0.5) |
| Medium follow-up (days) (IQR) | 77 (32) | 102 (32) |

Data are presented as n (%) unless otherwise noted.

Postoperative complications are listed in Table 3 below. Perioperative transient urinary retention (24 hours or less) was present in 8%. No one had retention longer than 24 hours. There were 10 episodes of post op UTI and 6 patients with de novo urgency symptoms. No other complications were noted. None of the patients came to receive a midurethral sling or other anti-incontinence procedure after Bulkamid. One patient had elected to have an incontinence pessary ring placed.

TABLE 3
Postoperative Complications

| | All participants | All participants |
|---|---|---|
| | (n = 126) | (n = 113) |
| Acute urinary retention | 10 (8) | 9 (8) |
| De novo UUI | 6 (5) | 5 (4) |
| Acute urinary tract infection | 10 (8) | 8 (7) |

Data are presented as n (%) unless otherwise noted.

While a method of treating stress urinary incontinence has been described in terms of what may be considered to be specific aspects, the present invention is not limited to the disclosed aspects. Additional modifications and improvements to the invention may be apparent to those skilled in the art. Moreover, the many features and advantages of the disclosure are apparent from the detailed specification, and thus, it is intended by the appended claims to cover all such features and advantages of the present invention which fall within the spirit and scope of the disclosure.

Further, it is not desired to limit the disclosure to the exact construction and operation illustrated and described, and accordingly, all suitable modifications and equivalents may be resorted to, falling within the scope of the disclosure. The present disclosure should therefore be considered as illustrative and not restrictive. As such, this disclosure is intended to cover various modifications and similar arrangements included within the spirit and scope of the claims, which should be accorded their broadest interpretation so as to encompass all such modifications and similar structures.

1. A method of treating stress urinary incontinence in a female subject, comprising administering a bulking agent into the external urethral sphincter of the subject.

2. (canceled)

3. The method of claim 1, wherein the bulking agent is administered into the external urethral sphincter of the subject at a position that is about 2-3 cm from the bladder neck.

4. The method of claim 1, wherein the bulking agent is administered into to the circumference of the external urethral sphincter.

5. (canceled)

6. (canceled)

7. (canceled)

8. The method of claim 1, wherein the bulking agent is a hydrogel.

9. (canceled)

10. The method of claim 8, wherein the hydrogel comprises polyacrylamide hydrogel (PAHG).

11. The method of claim 10, wherein the polyacrylamide comprises cross-linked polyacrylamide.

12. The method of claim 10, wherein said PAHG comprises about 1% to about 10% (w/w) polyacrylamide.

13. (canceled)

14. (canceled)

15. (canceled)

16. (canceled)

17. (canceled)

18. (canceled)

19. (canceled)

20. (canceled)

21. The method of claim 1, wherein about 1 to about 10 ml of bulking agent is administered into the external urethral sphincter of the subject.

22. (canceled)

23. The method of claim 1, wherein an amount of bulking agent sufficient to reconstruct the external urethral sphincter is administered into the external urethral sphincter of the subject.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method of treating stress urinary incontinence in a female subject, comprising performing an endoscopic reconstruction of the external urethral sphincter by injecting a bulking agent into the external urethral sphincter of the subject.

47. The method of claim 46, wherein the bulking agent is injected into the external urethral sphincter of the subject.

48. (canceled)

49. The method of claim 46, wherein the bulking agent is administered into to the circumference of the external urethral sphincter.

50. (canceled)

51. (canceled)

52. (canceled)

53. The method of claim 46, wherein the bulking agent comprises a hydrogel.

54. The method of claim 53, wherein the hydrogel comprises polyacrylamide hydrogel (PAHG).

55. (canceled)

56. The method of claim 54, wherein said PAHG comprises about 1% to about 10% (w/w) polyacrylamide.

57. (canceled)

58. (canceled)

59. (canceled)

60. (canceled)

61. (canceled)

62. (canceled)

63. (canceled)

64. (canceled)

65. The method of claim 46, wherein about 1 ml to about 10 ml of PAHG is administered into the external urethral sphincter of the subject.

66. (canceled)

67. (canceled)

68. A method of improving longevity of a treatment of treating stress urinary incontinence of a subject, comprising:
performing the method of claim 1; and
providing instructions to the subject for abstaining from pelvic muscle clenching and abdominal straining while voiding and/or defecating after the agent has been administered, thereby preventing dislodging of the administered agent.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. A kit for use in treating stress urinary incontinence in a female subject, the kit comprising one or more dispensing devices and a bulking agent for administering into the external urethral sphincter of the subject via at least one of the one or more dispensing devices.

83. (canceled)

84. The kit of claim 82, wherein the bulking agent comprises a hydrogel.

85. The kit of claim 82, wherein the bulking agent comprises a polyacrylamide hydrogel (PAHG).

86. (canceled)

87. (canceled)

88. (canceled)

89. (canceled)
